(19) United States
(12) Patent Application Publication
(10) Pub. No.: US 2022/0400669 A1
(43) Pub. Date: Dec. 22, 2022

(54) COMPOUNDS AND METHODS FOR PROMOTING PLANT GROWTH

(71) Applicants: King Abdullah University of Science and Technology, Thuwal (SA); University of Tokyo, Tokyo (JP)

(72) Inventors: Salim Albabili, Thuwal (SA); Tadao Asami, Tokyo (JP)

(21) Appl. No.: 17/771,717

(22) PCT Filed: Oct. 26, 2020
(86) PCT No.: PCT/IB2020/060033

§ 371 Date: Apr. 25, 2022

Related U.S. Application Data
(60) Provisional application No. 62/925,555, filed on Oct. 24, 2019.

(51) Int. Cl. A01N 31/04; C07C 49/217; C07C 49/248; C07C 49/255; A01N 31/14; A01N 31/16

(52) U.S. Cl. CPC A01N 31/04; C07C 49/217; C07C 49/248; C07C 49/255; A01N 31/14; A01N 31/16

(57) ABSTRACT

Disclosed herein are compounds and methods for promoting plant growth. Formulations containing one or more disclosed compounds or salts thereof, and one or more excipients are disclosed. The formulation adjuvant can be a solid carrier, a liquid carrier, or a surface-active agent. Methods for promoting plant growth typically includes applying one or more formulations to a plant, a plant part, or a growing site of plant. The plant can be a cereal, grain, or vegetable plant. The plant part can be seed or seedling of a plant. The growing site of plant can be soil before, during, or after the plant is planted or a growing medium. In some embodiments, the one or more compounds or salts thereof in the one or more formulations are in an effective amount to decrease biosynthesis and release of strigolactone plant hormones from the plant.

### CROSS-REFERENCE TO RELATED APPLICATIONS

This application claims the benefit of and priority to U.S. Provisional Application No. 62/925,555 filed Oct. 24, 2019, which is hereby incorporated by reference in its entirety.

### FIELD OF THE INVENTION

The invention is generally directed to compounds and methods for promoting plant growth.

### BACKGROUND OF THE INVENTION

Parasitic plants are plants that derive some or all of the necessary nutritional requirements from a living host plant. More than 4000 parasitic plants are known and these plants can cause a range of effects anywhere from minor damage to a host plant up to and including destruction of the host. In particular, root parasitic plants specifically parasitize the roots of the host plant. Seeds of the parasitic plants germinate in the soil near the host roots and use haustoria to penetrate the host roots to reach nutrients. Grain crops are among the plants that can be most severely affected by root parasitic plants.

Use of herbicides to control root parasitic plants has had limited success for a number of reasons. Herbicides that are effective on root parasitic weeds are typically also toxic to the desired plants, and selective herbicides that do not affect the desired plants are frequently not effective on the parasitic plants. In some areas, limited or uneven water availability punctuated by uncontrolled flooding further reduces the effectiveness of herbicides.

One means of combating root parasitic plants, such as Striga, is the application of strigolactone analogs to soil where seeds of the parasite reside. Application of a strigolactone analog in the absence of a suitable host plant induces germination of the parasitic plant's seeds; however, the resulting seedlings are unable to tap into a host plant's roots to acquire the necessary nutrients and will die out before emerging from the soil. Since the parasitic plant would be unable to complete the growth cycle, no new seeds will be produced to remain in the soil. This “suicide germination” approach is a promising approach to combat root parasitic weeds. However, it is effective only if there is adequate time to treat the soil prior to planting a desired cereal, grain, or vegetable crops, and would not be effective during the growth cycle of the desired crops.

Another approach has been the development of plant varieties that are resistant to root parasitic weeds. Pearl millet accessions contrasting for Striga resistance are currently being characterized and show promise. First results point to a role of SLs release, among other factors. However, the process of developing new resistant strains for the many types of affected host plants could take years to complete.

There remains a need for effective means to combat root parasitic weeds that is compatible with growing the desired cereal, grain and vegetable crops.

Therefore, it is the object of the present invention to provide compounds for promoting plant growth.

It is another object of the present invention to provide methods of making such compounds.

It is yet another object of the present invention to provide methods of using such compounds.

### SUMMARY OF THE INVENTION

Disclosed herein are compounds having a structure of Formula I:

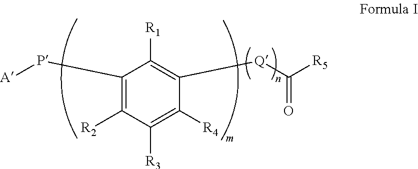

(a) where A′ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group;

(b) where P′ is an oxygen atom, a sulfur atom, or absent;

(c) where m is zero or a positive integer;

(d) where Q′ is —CR₆═CR₇—.

(e) where n is zero or a positive integer; and

(f) where R₁-R₇, when present, are independently

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,

an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonyl group containing one substituent, a carbonate ester group containing one substituent, an ether group containing one substituent, an aminooxy group optionally containing one or two substituents at the amino nitrogen, or a hydroxyamino group optionally containing one or two substituents,

where the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

In some embodiments, m is a positive integer.

Formulations containing one or more compounds of Formula (I) or salts thereof, and one or more excipients are disclosed.

The formulation excipient can be a solid carrier, a liquid carrier, or a surface-active agent.

The formulation can be in the form of dusting powders, gels, wettable powders, water-dispersible granules, water-dispersible tablets, effervescent compressed tablets, emulsifiable concentrates, microemulsifiable concentrates, oil-in-water emulsions, oil flowables, aqueous dispersions, oil dispersions, suspoemulsions capsule suspensions, emulsifiable granules, soluble liquids, water-soluble concentrates (with water or a water miscible organic solvent as carrier), or impregnated polymer films.

Methods for using the one or more compounds or salts thereof, and formulations are also disclosed. The compounds and formulations are used to promote plant growth.

A method for promoting plant growth typically includes applying one or more formulations to a plant, a plant part, or a growing site of plant, where the one or more compounds or salts thereof in the one or more formulations are in an effective amount to promote plant growth.

The plant can be a cereal, grain, or vegetable plant. The plant part can be seed or seedling of the plant.

In some embodiments, the one or more compounds or salts thereof in the formulations are in an effective amount to decrease biosynthesis and release of strigolactone plant hormones from the plant. Optionally, the biosynthesis and release of strigolactone plant hormones from the plant is decreased by at least about 40% compared to an untreated plant under same conditions.

A method for inhibiting growth of a parasitic weed typically includes applying one or more formulations to the parasitic weed, a part of the parasitic weed, or a growing site of parasitic weed, where the one or more compounds or salts thereof are in an effective amount to inhibit growth of the parasitic weed.

The parasitic weed can be a Striga species or an Orobanche species of the Orobanchaceae family.

In some embodiments, the one or more compounds or salts thereof in the formulations are in an effective amount to inhibit germination of a seed of the parasitic weed.

### BRIEF DESCRIPTION OF THE DRAWINGS

FIGS. 1A-1D are bar graphs showing the qRT-PCR analysis of transcript levels of SL biosynthesis genes β-carotene cis-trans isomerase Dwarf27 (D27) (FIG. 1A), Carotenoid Cleavage Dioxygenase 7 (CCD7) (FIG. 1B), Carotenoid Cleavage Dioxygenase 8 (CCD8) (FIG. 1C), and carlactone oxidase (OsCO) (FIG. 1D). Transcript levels in wild-type control samples (CTL) were normalized to 1. Bars represent mean±SD; n=3 biological replicates. Statistical analysis was performed using One-way analysis of variance (ANOVA) and Tukey's post hoc test. Different letters denote significant differences (P<0.05).

FIG. 2 is a graph showing Striga seeds germination rate upon treatment with Zaxinone (Zax) and MZ2-MZ5 compounds.

FIG. 3 is a bar graph showing stability of MZ compounds and Zax by HPLC analysis. The relative amount of non-degraded analogs was monitored in HPLC daily up to 14 days and calculated by comparison with internal standard. Data are means±SE (n=3). X-axis: time (days); Y-axis: relative levels. Statistical analysis was performed using One-way analysis of variance (ANOVA) and Tukey's post hoc test. Different letters denote significant differences (P<0.05). CTL represents control samples.

FIGS. 4A-4C are graphs showing root length (FIG. 4A), root biomass (FIG. 4B), and crown root number (FIG. 4C) of hydroponically grown wild-type rice and zas mutant rice seedlings in the absence (Control) and presence of Zax, MZ3, and MZ5 at 2.5 μM.

FIGS. 5A-5B are graphs showing effect of Zax, MZ3, and MZ5 at 5 μM on the root surface area (FIG. 5A) and root numbers (FIG. 5B) of soil-grown (rhizotron) wild-type rice plants.

FIGS. 6A-6B are graphs showing tillering phenotype of WT rice seedlings (FIG. 6A) and IAC165 rice cultivar seedlings (FIG. 6B) in response to Zax, MZ3, and MZ5 (2.5 μM).

FIGS. 7A-7D are bar graphs showing foliar application of Zax, MZ3, and MZ5 at 5 μM on the height (FIG. 7A), number of branches per plant (FIG. 7B), number of flowers per plant (FIG. 7C), and number of fruits per plant (FIG. 7D) of tomato plants. Each data point represents one plant [(a), n=6; (b), n=5; (c), n=7; (d), n=5]. Data represent mean±SD. Statistical analysis was performed using One-way analysis of variance (ANOVA) and Tukey's post hoc test and t-test. Different letters denote significant differences (P<0.05). CTL represents control (no treatment).

FIGS. 8A-8B are bar graphs showing the quantification of 4-deoxyorobanchol (4DO) (FIG. 8A) and Orobanchol (Oro) (FIG. 8B) in wild-type rice roots in response to Zax, MZ3, and MZ5 at 5 μM under Pi starvation.

FIGS. 9A-9B are bar graphs showing the quantification of 4-deoxyorobanchol (4DO) (FIG. 9A) and Orobanchol (Oro) (FIG. 9B) in wild-type root exudates in response to Zax, MZ3, and MZ5 at 5 μM under Pi starvation.

FIG. 10 is a bar graph showing Striga seeds germination rate upon treatment with exudates analyzed in FIGS. 9A and 9B.

FIGS. 11A and 11B are bar graphs showing quantification of 4-deoxyorobanchol (4DO) in wild-type rice and zas mutant rice root exudates (FIG. 11A) and roots (FIG. 11B) in response to Zax, MZ3, and MZ5 at 5 μM under Pi starvation. Bars represent mean±SD; n=4 biological replicates; statistical analysis was performed using One-way analysis of variance (ANOVA) and Tukey's post hoc test. Different letters denote significant differences (P<0.05).

FIGS. 12A-12D are bar graphs showing qRT-PCR analysis of transcript levels of SL biosynthesis genes D27 (FIG. 12A), CCD7 (FIG. 12B), CCD8 (FIG. 12C), and CO (FIG. 12D) analyzed in FIGS. 8A and 8B. Transcript levels in wild-type control samples were normalized to 1. Bars represent mean±SD; n=4 biological replicates. Different letters denote significant differences (P<0.05).

FIG. 13 is a bar graph showing effect of Zax, MZ3, and MZ5 (5 μM) on Striga infestation of six-week old rice vs. IAC-165 plants in soil. Bars represent mean±SE; n=4 biological replicates. Statistical analysis was performed using One-way analysis of variance (ANOVA) and Tukey's post hoc test. Different letters denote significant differences (P<0.05).

FIGS. 14A-14B are graphs showing effect of MZ3 and MZ5 on Gigaspora margarita spores germination after 3 (FIG. 14A) and 7 (FIG. 14B) days incubation. Bars represent mean±SE; n=96 biological replicates; Data indicated with different letters are statistically different according to the non-parametric Kruskal-Wallis test. Different letters denote significant differences (P<0.05). FIG. 14C is a bar graph showing effect of MZ compounds on SL-mediated Gigaspora margarita spore germination. Bars represent mean±SE; n=96 biological replicates; Data indicated with different letters are statistically different according to the non-parametric Kruskal-Wallis test (p<0.05). CLT: spores treated with acetone; GR24 (SL analog): spores treated with GR24 (10 nM); MZ3: spores treated with MZ3 (5 μM); MZ3/GR24: spores treated with MZ3 (5 μM) and GR24 (10 nM); MZ5: spores treated with MZ5 (5 μM); MZ5/GR24: spores treated with MZ5 (5 μM) and GR24 (10 nM).

FIGS. 15A-15B are graphs showing effect of Zax on Gigaspora margarita spores germination after 3 (FIG. 15A) and 7 (FIG. 15B) days incubation, n=96 biological replicates; Data indicated with different letters are statistically different according to the non-parametric Kruskal-Wallis test (p<0.05). CTL, Control; Zax, Zaxinone.

FIGS. 16A-16C are bar graphs showing mycorrhizal colonization in non-treated and treated plants by the AM fungi Funneliformis mosseae at 35 dpi: degree of colonization expressed as mycorrhizal frequency (FIG. 16A), mycorrhizal intensity (FIG. 16B), and arbuscule abundance (FIG. 16C) in the root system of WT plants non treated and treated with 5 μM and 50 nM MZ3 or MZ5. Data are the average of four biological replicates ±SE. CTL, Control; N.S., non-significant. FIG. 16D-16F are bar graphs showing molecular evaluation of mycorrhization by qRT-PCR analysis of mRNA abundance of plant AM-responsive genes (OsPT11, OsLysM) (FIGS. 16D and 16E) and fungal housekeeping gene (18S) (FIG. 16F) on mycorrhizal roots considering the whole root system (large lateral roots and crown roots). Bars represent mean±SD; n=4 biological replicates, Statistical analysis was performed using One-way analysis of variance (ANOVA) and Tukey's post hoc test.

FIGS. 17A-17D are bar graphs showing phenotypic evaluation (root length (FIG. 17A and FIG. 17C); crown root number (FIGS. 17B and 17D)) of different concentrations of MZ compounds on non-mycorrhizal plants. The non-mycorrhizal plants were grown in sand and watered with Long Ashton solution (3.2 μM Pi) for five weeks post AM fungus inoculation. Twice a week treatment with MZ compounds at 5 μM or 50 μM increased the crown root length in non-mycorrhizal plants.

FIGS. 18A-18D are bar graphs showing phenotypic evaluation (root length (FIGS. 18A and 18C); crown root number (FIGS. 18B and 18D)) of different concentrations of MZ compounds on mycorrhizal plants. The mycorrhizal plants were grown in sand and watered with Long Ashton solution (3.2 μM Pi) for five weeks post AM fungus inoculation. Twice a week treatment with MZ compounds at 5 μM or 50 μM increased the crown root number in mycorrhizal plants.

FIG. 19 is a bar showing the content of SLs treated with different MZ compounds (MZ1-MZ7).

FIGS. 20A-20B are graphs showing quantification of SLs, 4-deoxyorobanchol (4-DO) (FIGS. 20A-20B) and Orobanchol (Oro) (FIG. 20B), in wild-type root exudates in response to zaxinone, and MZ1 at 5 μM under Pi starvation. Bars represent mean±SD; n=3 biological replicates; statistical analysis was performed using t-test.

FIGS. 21A-21B are bar graphs showing the quantification of SLs, 4-DO (FIG. 21A) and Oro (FIG. 21B), in wild-type root exudates in response to MZ1, MZ2, or MZ4 at 5 μM under Pi starvation. FIG. 21C is a bar graph showing the Striga seed germination activity of rice root exudates isolated from plants treated with MZ, MZ2, or MZ4 at 5 μM under Pi starvation. Bars represent mean±SD; n=3 biological replicates; statistical analysis was performed using t-test. *, P<0.05.

FIGS. 22A-22I are graphs showing the height of the green pepper plant in fresh ground water (FIG. 22A), medium ground salt water (FIG. 22B), and high salt (FIG. 22C); the number of leaves of the green pepper plant in fresh ground water (FIG. 22D), medium ground salt water (FIG. 22E), and high salt (FIG. 22F); and the number of branches of the green pepper plant in fresh ground water (FIG. 22G), medium ground salt water (FIG. 22H), and high salt (FIG. 22I).

FIGS. 23A-23I are graphs showing the number of flower of the green pepper plant in fresh ground water (FIG. 23A), medium ground salt water (FIG. 23B), and high salt (FIG. 23C); the plant fresh weight of the green pepper plant in fresh ground water (FIG. 23D), medium ground salt water (FIG. 23E), and high salt (FIG. 23F); and the SPD of the green pepper plant in fresh ground water (FIG. 23G), medium ground salt water (FIG. 23H), and high salt (FIG. 23I).

FIGS. 24A-24I are graphs showing the root length of the green pepper plant in fresh ground water (FIG. 24A), medium ground salt water (FIG. 24B), and high salt (FIG. 24C); the root fresh weight of the green pepper plant in fresh ground water (FIG. 24D), medium ground salt water (FIG. 24E), and high salt (FIG. 24F); and the individual leave area of the green pepper plant in fresh ground water (FIG. 24G), medium ground salt water (FIG. 24H), and high salt (FIG. 24I).

FIGS. 25A-25I are graphs showing the number of fruit of the green pepper plant in fresh ground water (FIG. 25A), medium ground salt water (FIG. 25B), and high salt (FIG. 25C); the average weight of single fruit of the green pepper plant in fresh ground water (FIG. 25D), medium ground salt water (FIG. 25E) and high salt (FIG. 25F); and the yield of the green pepper plant in fresh ground water (FIG. 25G), medium ground salt water (FIG. 25H), and high salt (FIG. 25I).

FIGS. 26A-26I are graphs showing the fruit length of the green pepper plant in fresh ground water (FIG. 26A), medium ground salt water (FIG. 26B), and high salt (FIG. 26C); the fruit diameter of the green pepper plant in fresh ground water (FIG. 26D), medium ground salt water (FIG. 26E), and high salt (FIG. 26F); and the fruit firmness of the green pepper plant in fresh ground water (FIG. 26G), medium ground salt water (FIG. 26H), and high salt (FIG. 26I).

FIGS. 27A-27F are graphs showing the fruit acidity of the green pepper plant in fresh ground water (FIG. 27A), medium ground salt water (FIG. 27B), and high salt (FIG. 27C); and the fruit total dissolfided solids of the green pepper plant in fresh ground water (FIG. 27D), medium ground salt water (FIG. 27E), and high salt (FIG. 27F).

FIGS. 28A-28F are graphs showing the fruit vitamin C content of the green pepper plant in fresh ground water (FIG. 28A), medium ground salt water (FIG. 28B), and high salt (FIG. 28C); and the fruit phenol content of the green pepper plant in fresh ground water (FIG. 28D), medium ground salt water (FIG. 28E), and high salt (FIG. 28F).

### DETAILED DESCRIPTION OF THE INVENTION

### I. Compositions

A. Compounds

Disclosed herein are compounds that promote plant growth. In particular, the disclosed compounds have structures of Formula I or salt thereof.

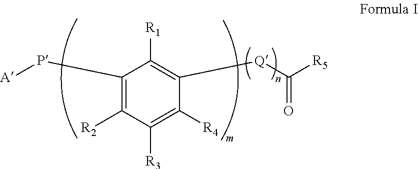

where A′ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group;

where P′ is an oxygen atom, a sulfur atom, —NH, or absent;

where m is zero or a positive integer;

where Q′ is —CR₆≡CR₇—;

where n is zero or a positive integer; and

where R₁-R₇, when present, are independently

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,

an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonyl group containing one substituent, a carbonate ester group containing one substituent, an ether group containing one substituent, an aminooxy group optionally containing one or two substituents at the amino nitrogen, or a hydroxyamino group optionally containing one or two substituents,

where the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

The alkyl group can be linear, branched, or cyclic. An alkyl can be a linear C₁-C₃₀ alkyl, a branched C₄-C₃₀ alkyl, a cyclic C₃-C₃₀ alkyl, a linear C₁-C₃₀ alkyl or a branched C₄-C₃₀ alkyl, a linear C₁-C₃₀ alkyl or a cyclic C₃-C₃₀ alkyl, a branched C₄-C₃₀ alkyl or a cyclic C₃-C₃₀ alkyl. Optionally, alkyl groups have up to 20 carbon atoms. An alkyl can be a linear C₁-C₂₀ alkyl, a branched C₄-C₂₀ alkyl, a cyclic C₃-C₂₀ alkyl, a linear C₁-C₂₀ alkyl or a branched C₄-C₂₀ alkyl, a branched C₄-C₂₀ alkyl or a cyclic C₃-C₂₀ alkyl, a linear C₁-C₂₀ alkyl or a cyclic C₃-C₂₀ alkyl. Optionally, alkyl groups have up to 10 carbon atoms. An alkyl can be a linear C₁-C₁₀ alkyl, a branched C₄-C₁₀ alkyl, a cyclic C₃-C₁₀ alkyl, a linear C₁-C₁₀ alkyl or a branched C₄-C₁₀ alkyl, a branched C₄-C₁₀ alkyl or a cyclic C₃-C₁₀ alkyl, a linear C₁-C₁₀ alkyl or a cyclic C₃-C₁₀ alkyl. Optionally, alkyl groups have up to 6 carbon atoms. An alkyl can be a linear C₁-C₆ alkyl, a branched C₄-C₆ alkyl, a cyclic C₃-C₆ alkyl, a linear C₁-C₆ alkyl or a branched C₄-C₆ alkyl, a branched C₄-C₆ alkyl or a cyclic C₃-C₆ alkyl, or a linear C₁-C₆ alkyl or a cyclic C₃-C₆ alkyl. Optionally, alkyl groups have up to four carbons. An alkyl can be a linear C₁-C₄ alkyl, cyclic C₃-C₄ alkyl, a linear C₁-C₄ alkyl or a cyclic C₃-C₄ alkyl.

The heteroalkyl group can be linear, branched, or cyclic. A heteroalkyl can be a linear C₁-C₃₀ heteroalkyl, a branched C₃-C₃₀ heteroalkyl, a cyclic C₂-C₃₀ heteroalkyl, a linear C₁-C₃₀ heteroalkyl or a branched C₃-C₃₀ heteroalkyl, a linear C₁-C₃₀ heteroalkyl or a cyclic C₂-C₃₀ heteroalkyl, a branched C₃-C₃₀ heteroalkyl or a cyclic C₂-C₃₀ heteroalkyl. Optionally, heteroalkyl groups have up to 20 carbon atoms. A heteroalkyl can be a linear C₁-C₂₀ heteroalkyl, a branched C₃-C₂₀ heteroalkyl, a cyclic C₂-C₂₀ heteroalkyl, a linear C₁-C₂₀ heteroalkyl or a branched C₃-C₂₀ heteroalkyl, a branched C₃-C₂₀ heteroalkyl or a cyclic C₂-C₂₀ heteroalkyl, or a linear C₁-C₂₀ heteroalkyl or a cyclic C₂-C₂₀ heteroalkyl. Optionally, heteroalkyl groups have up to 10 carbon atoms. A heteroalkyl can be a linear C₁-C₁₀ heteroalkyl, a branched C₃-C₁₀ heteroalkyl, a cyclic C₂-C₁₀ heteroalkyl, a linear C₁-C₁₀ heteroalkyl or a branched C₃-C₁₀ heteroalkyl, a branched C₃-C₁₀ heteroalkyl or a cyclic C₂-C₁₀ heteroalkyl, or a linear C₁-C₁₀ heteroalkyl or a cyclic C₂-C₁₀ heteroalkyl. Optionally, heteroalkyl groups have up to 6 carbon atoms. A heteroalkyl can be a linear C₁-C₆ heteroalkyl, a branched C₃-C₆ heteroalkyl, a cyclic C₂-C₆ heteroalkyl, a linear C₁-C₆ heteroalkyl or a branched C₃-C₆ heteroalkyl, a branched C₃-C₆ heteroalkyl or a cyclic C₂-C₆ heteroalkyl, or a linear C₁-C₆ heteroalkyl or a cyclic C₂-C₆ heteroalkyl. Optionally, heteroalkyl groups have up to four carbons. A heteroalkyl can be a linear C₁-C₄ heteroalkyl, a branched C₃-C₄ heteroalkyl, a cyclic C₂-C₄ heteroalkyl, a linear C₁-C₄ heteroalkyl or a branched C₃-C₄ heteroalkyl, a branched C₃-C₄ heteroalkyl or a cyclic C₂-C₄ heteroalkyl, or a linear C₁-C₄ heteroalkyl or a cyclic C₂-C₄ heteroalkyl.

The alkenyl group can be linear, branched, or cyclic. An alkenyl can be a linear C₂-C₃₀ alkenyl, a branched C₄-C₃₀ alkenyl, a cyclic C₃-C₃₀ alkenyl, a linear C₂-C₃₀ alkenyl or a branched C₄-C₃₀ alkenyl, a linear C₂-C₃₀ alkenyl or a cyclic C₃-C₃₀ alkenyl, a branched C₄-C₃₀ alkenyl or a cyclic C₃-C₃₀ alkenyl. Optionally, alkenyl groups have up to 20 carbon atoms. An alkenyl can be a linear C₂-C₂₀ alkenyl, a branched C₄-C₂₀ alkenyl, a cyclic C₃-C₂₀ alkenyl, a linear C₂-C₂₀ alkenyl or a branched C₄-C₂₀ alkenyl, a linear C₂-C₂₀ alkenyl or a cyclic C₃-C₂₀ alkenyl, a branched C₄-C₂₀ alkenyl or a cyclic C₃-C₂₀ alkenyl. Optionally, alkenyl groups have two to 10 carbon atoms. An alkenyl can be a linear C₂-C₁₀ alkenyl, a branched C₄-C₁₀ alkenyl, a cyclic C₃-C₁₀ alkenyl, a linear C₂-C₁₀ alkenyl or a branched C₄-C₁₀ alkenyl, a linear C₂-C₁₀ alkenyl or a cyclic C₃-C₁₀ alkenyl, a branched C₄-C₁₀ alkenyl or a cyclic C₃-C₁₀ alkenyl. Optionally, alkenyl groups have two to 6 carbon atoms. An alkenyl can be a linear C₂-C₆ alkenyl, a branched C₄-C₆ alkenyl, a cyclic C₃-C₆ alkenyl, a linear C₂-C₆ alkenyl or a branched C₄-C₆ alkenyl, a linear C₂-C₆ alkenyl or a cyclic C₃-C₆ alkenyl, a branched C₄-C₆ alkenyl or a cyclic C₃-C₆ alkenyl. Optionally, alkenyl groups have two to four carbons. An alkenyl can be a linear C₂-C₄ alkenyl, a cyclic C₃-C₄ alkenyl, a linear C₂-C₄ alkenyl or a cyclic C₃-C₄ alkenyl.

The heteroalkenyl group can be linear, branched, or cyclic. A heteroalkenyl can be a linear C₂-C₃₀ heteroalkenyl, a branched C₃-C₃₀ heteroalkenyl, a cyclic C₂-C₃₀ heteroalkenyl, a linear C₂-C₃₀ heteroalkenyl or a branched C₃-C₃₀ heteroalkenyl, a linear C₂-C₃₀ heteroalkenyl or a cyclic C₂-C₃₀ heteroalkenyl, a branched C₃-C₃₀ heteroalkenyl or a cyclic C₂-C₃₀ heteroalkenyl. Optionally, heteroalkenyl groups have up to 20 carbon atoms. A heteroalkenyl can be a linear C₂-C₂₀ heteroalkenyl, a branched C₃-C₂₀ heteroalkenyl, a cyclic C₂-C₂₀ heteroalkenyl, a linear C₂-C₂₀ heteroalkenyl or a branched C₃-C₂₀ heteroalkenyl, a linear C₂-C₂₀ heteroalkenyl or a cyclic C₂-C₂₀ heteroalkenyl, a branched C₃-C₂₀ heteroalkenyl or a cyclic C₂-C₂₀ heteroalkenyl. Optionally, heteroalkenyl groups have up to 10 carbon atoms. A heteroalkenyl can be a linear C₂-C₁₀ heteroalkenyl, a branched C₃-C₁₀ heteroalkenyl, a cyclic C₂-C₁₀ heteroalkenyl, a linear C₂-C₁₀ heteroalkenyl or a branched C₃-C₁₀ heteroalkenyl, a linear C₂-C₁₀ heteroalkenyl or a cyclic C₂-C₁₀ heteroalkenyl, a branched C₃-C₁₀ heteroalkenyl or a cyclic C₂-C₁₀ heteroalkenyl. Optionally, heteroalkenyl groups have two to 6 carbon atoms. A heteroalkenyl can be a linear C₂-C₆ heteroalkenyl, a branched C₃-C₆ heteroalkenyl, a cyclic C₂-C₆ heteroalkenyl, a linear C₂-C₆ heteroalkenyl or a branched C₃-C₆ heteroalkenyl, a linear C₂-C₆ heteroalkenyl or a cyclic C₂-C₆ heteroalkenyl, a branched C₃-C₆ heteroalkenyl or a cyclic C₂-C₆ heteroalkenyl. Optionally, heteroalkenyl groups have two to four carbons. A heteroalkenyl can be a linear C₂-C₄ heteroalkenyl, a branched C₃-C₄ heteroalkenyl, a cyclic C₂-C₄ heteroalkenyl, a linear C₂-C₄ heteroalkenyl or a branched C₃-C₄ heteroalkenyl, a linear C₂-C₄ heteroalkenyl or a cyclic C₂-C₄ heteroalkenyl, a branched C₃-C₄ heteroalkenyl or a cyclic C₂-C₄ heteroalkenyl.

The alkynyl group can be linear, branched, or cyclic. An alkynyl can be a linear C₂-C₃₀ alkynyl, a branched C₄-C₃₀ alkynyl, a cyclic C₃-C₃₀ alkynyl, a linear C₂-C₃₀ alkynyl or a branched C₄-C₃₀ alkynyl, a linear C₂-C₃₀ alkynyl or a cyclic C₃-C₃₀ alkynyl, a branched C₄-C₃₀ alkynyl or a cyclic C₃-C₃₀ alkynyl. Optionally, alkynyl groups have up to 20 carbon atoms. An alkynyl can be a linear C₂-C₂₀ alkynyl, a branched C₄-C₂₀ alkynyl, a cyclic C₃-C₂₀ alkynyl, a linear C₂-C₂₀ alkynyl or a branched C₄-C₂₀ alkynyl, a branched C₄-C₂₀ alkynyl or a cyclic C₃-C₂₀ alkynyl. Optionally, alkynyl groups have up to 10 carbon atoms. An alkynyl can be a linear C₂-C₁₀ alkynyl, a branched C₄-C₁₀ alkynyl, a cyclic C₃-C₁₀ alkynyl, a linear C₂-C₂₀ alkynyl or a branched C₄-C₁₀ alkynyl, a branched C₄-C₂₀ alkynyl or a cyclic C₃-C₁₀ alkynyl, a linear C₂-C₂₀ alkynyl or a cyclic C₃-C₂₀ alkynyl. Optionally, alkynyl groups have up to 6 carbon atoms. An alkynyl can be a linear C₂-C₆ alkynyl, a branched C₄-C₆ alkynyl, a cyclic C₃-C₆ alkynyl, a linear C₂-C₆ alkynyl or a branched C₄-C₆ alkynyl, a branched C₄-C₆ alkynyl or a cyclic C₃-C₆ alkynyl, a linear C₂-C₆ alkynyl or a cyclic C₃-C₆ alkynyl. Optionally, alkynyl groups have up to four carbons. An alkynyl can be a linear C₂-C₄ alkynyl, a cyclic C₃-C₄ alkynyl, a linear C₂-C₄ alkynyl or a cyclic C₃-C₄ alkynyl.

The heteroalkynyl group can be linear, branched, or cyclic. A heteroalkynyl can be a linear C₂-C₃₀ heteroalkynyl, a branched C₃-C₃₀ heteroalkynyl, a cyclic C₂-C₃₀ heteroalkynyl, a linear C₂-C₃₀ heteroalkynyl or a branched C₃-C₃₀ heteroalkynyl, a linear C₂-C₃₀ heteroalkynyl or a cyclic C₂-C₃₀ heteroalkynyl, a branched C₃-C₃₀ heteroalkynyl or a cyclic C₂-C₃₀ heteroalkynyl. Optionally, heteroalkynyl groups have up to 20 carbon atoms. A heteroalkynyl can be a linear C₂-C₂₀ heteroalkynyl, a branched C₃-C₂₀ heteroalkynyl, a cyclic C₂-C₂₀ heteroalkynyl, a linear C₂-C₂₀ heteroalkynyl or a branched C₃-C₂₀ heteroalkynyl, a branched C₃-C₂₀ heteroalkynyl or a cyclic C₂-C₂₀ heteroalkynyl, a linear C₂-C₂₀ heteroalkynyl or a cyclic C₂-C₂₀ heteroalkynyl. Optionally, heteroalkynyl groups have up to 10 carbon atoms. A heteroalkynyl can be a linear C₂-C₁₀ heteroalkynyl, a branched C₃-C₁₀ heteroalkynyl, a cyclic C₂-C₁₀ heteroalkynyl, a linear C₂-C₁₀ heteroalkynyl or a branched C₃-C₁₀ heteroalkynyl, a branched C₃-C₁₀ heteroalkynyl or a cyclic C₂-C₁₀ heteroalkynyl, a linear C₂-C₁₀ heteroalkynyl or a cyclic C₂-C₁₀ heteroalkynyl. Optionally, heteroalkynyl groups have two to 6 carbon atoms. A heteroalkynyl can be a linear C₂-C₆ heteroalkynyl, a branched C₃-C₆ heteroalkynyl, a cyclic C₂-C₆ heteroalkynyl, a linear C₂-C₆ heteroalkynyl or a branched C₃-C₆ heteroalkynyl, a branched C₃-C₆ heteroalkynyl or a cyclic C₂-C₆ heteroalkynyl, a linear C₂-C₆ heteroalkynyl or a cyclic C₂-C₆ heteroalkynyl. Optionally, heteroalkynyl groups have up to four carbons. A heteroalkynyl can be a linear C₂-C₄ heteroalkynyl, a branched C₃-C₄ heteroalkynyl, a cyclic C₂-C₄ heteroalkynyl, a linear C₂-C₄ heteroalkynyl or a branched C₃-C₄ heteroalkynyl, a branched C₃-C₄ heteroalkynyl or a cyclic C₂-C₄ heteroalkynyl, a linear C₂-C₄ heteroalkynyl or a cyclic C₂-C₄ heteroalkynyl.

The aryl group can have six to 50 carbon atoms. An aryl can be a branched C₆-C₅₀ aryl, a monocyclic C₆-C₅₀ aryl, a polycyclic C₆-C₅₀ aryl, a branched polycyclic C₆-C₅₀ aryl, a fused polycyclic C₆-C₅₀ aryl, or a branched fused polycyclic C₆-C₅₀ aryl. Optionally, aryl groups have six to 30 carbon atoms, i.e., C₆-C₃₀ aryl. A C₆-C₃₀ aryl can be a branched C₆-C₃₀ aryl, a monocyclic C₆-C₃₀ aryl, a polycyclic C₆-C₃₀ aryl, a branched polycyclic C₆-C₃₀ aryl, a fused polycyclic C₆-C₃₀ aryl, or a branched fused polycyclic C₆-C₃₀ aryl. Optionally, aryl groups have six to 20 carbon atoms, i.e., C₆-C₂₀ aryl. A C₆-C₂₀ aryl can be a branched C₆-C₂₀ aryl, a monocyclic C₆-C₂₀ aryl, a polycyclic C₆-C₂₀ aryl, a branched polycyclic C₆-C₂₀ aryl, a fused polycyclic C₆-C₂₀ aryl, or a branched fused polycyclic C₆-C₂₀ aryl. Optionally, aryl groups have six to twelve carbon atoms, i.e., C₆-C₁₂ aryl. A C₆-C₁₂ aryl can be a branched C₆-C₁₂ aryl, a monocyclic C₆-C₁₂ aryl, a polycyclic C₆-C₁₂ aryl, a branched polycyclic C₆-C₁₂ aryl, a fused polycyclic C₆-C₁₂ aryl, or a branched fused polycyclic C₆-C₁₂ aryl. Optionally, C₆-C₁₂ aryl groups have six to eleven carbon atoms, i.e., C₆-C₁₁ aryl. A C₆-C₁₁ aryl can be a branched C₆-C₁₁ aryl, a monocyclic C₆-C₁₁ aryl, a polycyclic C₆-C₁₁ aryl, a branched polycyclic C₆-C₁₁ aryl, a fused polycyclic C₆-C₁₁ aryl, or a branched fused polycyclic C₆-C₁₁ aryl. Optionally, C₆-C₁₂ aryl groups have six to nine carbon atoms, i.e., C₆-C₉ aryl. A C₆-C₉ aryl can be a branched C₆-C₉ aryl, a monocyclic C₆-C₉ aryl, a polycyclic C₆-C₉ aryl, a branched polycyclic C₆-C₉ aryl, a fused polycyclic C₆-C₉ aryl, or a branched fused polycyclic C₆-C₉ aryl. Optionally, C₆-C₁₂ aryl groups have six carbon atoms, i.e., C₆ aryl. A C₆ aryl can be a branched C₆ aryl or a monocyclic C₆ aryl.

The heteroaryl group can have three to 50 carbon atoms, i.e., C₃-C₅₀ heteroaryl. A C₃-C₅₀ heteroaryl can be a branched C₃-C₅₀ heteroaryl, a monocyclic C₃-C₅₀ heteroaryl, a polycyclic C₃-C₅₀ heteroaryl, a branched polycyclic C₃-C₅₀ heteroaryl, a fused polycyclic C₃-C₅₀ heteroaryl, or a branched fused polycyclic C₃-C₅₀ heteroaryl. Optionally, heteroaryl groups have six to 30 carbon atoms, i.e., C₆-C₃₀ heteroaryl. A C₆-C₃₀ heteroaryl can be a branched C₆-C₃₀ heteroaryl, a monocyclic C₆-C₃₀ heteroaryl, a polycyclic C₆-C₃₀ heteroaryl, a branched polycyclic C₆-C₃₀ heteroaryl, a fused polycyclic C₆-C₃₀ heteroaryl, or a branched fused polycyclic C₆-C₃₀ heteroaryl. Optionally, heteroaryl groups have six to 20 carbon atoms, i.e., C₆-C₂₀ heteroaryl. A C₆-C₂₀ heteroaryl can be a branched C₆-C₂₀ heteroaryl, a monocyclic C₆-C₂₀ heteroaryl, a polycyclic C₆-C₂₀ heteroaryl, a branched polycyclic C₆-C₂₀ heteroaryl, a fused polycyclic C₆-C₂₀ heteroaryl, or a branched fused polycyclic C₆-C₂₀ heteroaryl. Optionally, heteroaryl groups have six to twelve carbon atoms, i.e., C₆-C₁₂ heteroaryl. A C₆-C₁₂ heteroaryl can be a branched C₆-C₁₂ heteroaryl, a monocyclic C₆-C₁₂ heteroaryl, a polycyclic C₆-C₁₂ heteroaryl, a branched polycyclic C₆-C₁₂ heteroaryl, a fused polycyclic C₆-C₁₂ heteroaryl, or a branched fused polycyclic C₆-C₁₂ heteroaryl. Optionally, C₆-C₁₂ heteroaryl groups have six to eleven carbon atoms, i.e., C₆-C₁₁ heteroaryl. A C₆-C₁₁ heteroaryl can be a branched C₆-C₁₁ heteroaryl, a monocyclic C₆-C₁₁ heteroaryl, a polycyclic C₆-C₁₁ heteroaryl, a branched polycyclic C₆-C₁₁ heteroaryl, a fused polycyclic C₆-C₁₁ heteroaryl, or a branched fused polycyclic C₆-C₁₁ heteroaryl. Optionally, C₆-C₁₂ heteroaryl groups have six to nine carbon atoms, i.e., C₆-C₉ heteroaryl. A C₆-C₉ heteroaryl can be a branched C₆-C₉ heteroaryl, a monocyclic C₆-C₉ heteroaryl, a polycyclic C₆-C₉ heteroaryl, a branched polycyclic C₆-C₉ heteroaryl, a fused polycyclic C₆-C₉ heteroaryl, or a branched fused polycyclic C₆-C₉ heteroaryl. Optionally, C₆-C₁₂ heteroaryl groups have six carbon atoms, i.e., C₆ heteroaryl. A C₆ heteroaryl can be a branched C₆ heteroaryl, a monocyclic C₆ heteroaryl, a polycyclic C₆ heteroaryl, a branched polycyclic C₆ heteroaryl, a fused polycyclic C₆ heteroaryl, or a branched fused polycyclic C₆ heteroaryl.

In a substituted group or moiety, one or more hydrogen atoms in the chemical group or moiety is replaced with one or more substituents. Any substitution is in accordance with permitted valence of the substituted atom and the substituent, and that the substitution results in a stable compound, i.e., a compound that does not spontaneously undergo transformation such as by rearrangement, cyclization, elimination, etc. Suitable substituents include, but are not limited to a halogen atom, an alkyl group, a cycloalkyl group, a heteroalkyl group, a cycloheteroalkyl group, an alkenyl group, a heteroalkenyl group, an alkynyl group, a heteroalkynyl group, an aryl group, a heteroaryl group, a polyaryl group, a polyheteroaryl group, —OH, —SH, —NH₂, —N₃, —OCN, —NCO, —ONO₂, —CN, —NC, —ONO, —CONH₂, —NO, —NO₂, —ONH₂, —SCN, —SNCS, —CF₃, —CH₂CF₃, —CH₂C₁, —CHCl₂, —CH₂NH₂, —NHCOH, —CHO, —COCl, —COF, —COBr, —COOH, —SO₃H, —CH₂SO₂CH₃, —PO₃H₂, —OPO₃H₂, —P(═O)(OR^{T1′})(OR^{T2′}), OP(═O)(OR^{T1′})(OR^{T2′}), —BR^{T1′}(OR^{T2′}), —B(OR^{T1′})(OR^{T2′}), or G′R^{T1′} in which -T′ is —O—, —S—, —NR^{T2′}—, —C(═O)—, —S(═O)—, —SO₂—, —C(═O)O—, —C(═O)NR^{T2′}—, —OC(═O)—, —NR^{T2′}C(═O)—, —OC(═O)O—, —OC(═O)NR^{T2′}—, —NR^{T2′}C(═O)O—, —NR^{T2′}C(═O)NR^{T3′}—, —C(═S)—, —C(═S)S—, —SC(═S)—, —SC(═S)S—, —C(═NR^{T2′})—, —C(═NR^{T2′})O—, —C(═NR^{T2′})NR^{T3′}—, —OC(═NR^{T2′})—, —NR^{T2′}C(═NR^{T3′}), —NR^{T2′}SO₂—, —C(═NR^{T2′})NR^{T3′}—, —OC(═NR^{T2′})—, —NR^{T2′}C(═NR^{T3′})—, —NR^{T2′}SO₂—, —NR^{T2′}SO₂NR^{T3′}—, —NR^{T2′}C(═S)—, —SC(═S)NR^{T2′}—, —NR^{T2′}C(═S)S—, —NR^{T2′}C(═S)NR^{T3′}—, —SC(═NR^{T2′})—, —C(═S)NR^{T2′}—, —OC(═S)NR^{T2′}—, —NR^{T2′}C(═S)O—, —SC(═O)NR^{T2′}—, —NR^{T2′}C(═O)S—, —C(═O)S—, —SC(═)—, —SC(═O)S—, —C(═S)O—, —OC(═S)—, —OC(═S)O—, —SO₂NR^{T2′}—, —BR^{T2′}—, or —PR^{T2′}—; where each occurrence of R^{T1′}, R^{T2′}, and R^{T3′} is, independently, a hydrogen atom, a halogen atom, an alkyl group, a heteroalkyl group, an alkenyl group, a heteroalkenyl group, an alkynyl group, a heteroalkynyl group, an aryl group, or a heteroaryl group.

In some embodiments, A′ is an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group.

In some embodiments, m is a positive integer.

In some embodiments, the compound can have the structures of Formula II:

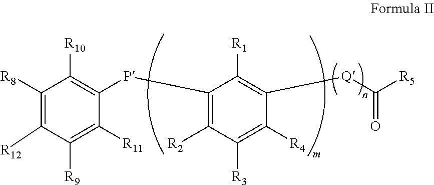

where P′, Q′, R₁-R₇, m, and n are as defined above; and

where R₈-R₁₂ are independently

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,

an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonyl group containing one substituent, a carbonate ester group containing one substituent, an ether group containing one substituent, an aminooxy group optionally containing one or two substituents at the amino nitrogen, or a hydroxyamino group optionally containing one or two substituents,

where the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

In some embodiments, R₁₂ is a hydrogen atom, an aldehyde group, an alkoxy group,

an amino group optionally containing one or two substituents at the amino nitrogen, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, an ether group containing one substituent, an aminooxy group optionally containing one or two substituents at the amino nitrogen, or a hydroxyamino group optionally containing one or two substituents,

where the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

Preferably, the length from P′ to R₅ is about the length of a C₈ chain.

In some embodiments of Formula II, P′ is an oxygen atom or absent.

In some embodiments of Formula II, P′ is an oxygen atom. In some embodiments of Formula II, P′ is absent.

In some embodiments of Formula II, m is a positive integer, and n is zero or a positive integer.

In some embodiments of Formula II, m and n are individually positive integers. In some embodiments of Formula II, m and n are individually positive integers between 1 and 10. In some embodiments of Formula II, m is a positive integer between 1 and 5, for example, 2, 4, or 5. In some embodiments of Formula II, n is a positive integer between 1 and 5, for example, 1 or 3. m and n can be the same or different. In some embodiments of Formula II, m is a positive integer and n is 1, for example, m is 1 and n is 1.

In some embodiments of Formula II, m is a positive integer, and n is zero. For example, m can be a positive integer and n can be zero. In some embodiments of Formula II, m is zero and n is a positive integer, for example, n is 3, 4, or 5, preferably 3.

In some embodiments of Formula II, when P′ is an oxygen atom, m and n are individually positive integers, for example, m is 1 and n is 1, or m is 2 and n is 1. In some embodiments of Formula II, P′ is absent. In some embodiments of Formula II, when P′ is absent, m is a positive integer, for example, 1, 2, 3, 4, or 5, and n is zero or a positive integer, for example, 1, 2, or 3. In some embodiments of Formula II, when P′ is absent, m is a positive integer and n is zero. In some embodiments of Formula II, when P′ is absent, m is a positive integer and n is 1. In some embodiments of Formula II, when P′ is absent, m is zero and n is a positive integer, for example, 3, 4, or 5, preferably 3.

In some embodiments of Formula II, R₁-R₇, when present, are independently a hydrogen atom, an unsubstituted alkyl group, a substituted alkyl group, an unsubstituted heteroalkyl group, or a substituted heteroalkyl group.

In some embodiments of Formula II, R₁-R₄, when present, are hydrogen and R₅-R₇, when present, are independently an unsubstituted alkyl group, a substituted alkyl group, an unsubstituted heteroalkyl group, or a substituted heteroalkyl group. In some embodiments of Formula II, R₁-R₄, when present, are hydrogen and R₅-R₇, when present, are independently an unsubstituted alkyl group or a substituted alkyl group. In some embodiments of Formula II, R₁-R₄, R₆, R₇, when present, are hydrogen, and R₅ is an unsubstituted alkyl group or a substituted alkyl group, for example, a methyl group.

In some embodiments of Formula II, R₈-R₁₁ are independently a hydrogen atom, an unsubstituted alkyl group, a substituted alkyl group, an unsubstituted heteroalkyl group, or a substituted heteroalkyl group. In some embodiments of Formula II, R₈-R₁₁ are independently a hydrogen atom, an unsubstituted alkyl group or a substituted alkyl group, for example, a methyl group. In some embodiments of Formula II, R₈ and R₉ are hydrogen, R₁₀ and R₁₁ are independently an unsubstituted alkyl group, a substituted alkyl group, an unsubstituted heteroalkyl group, or a substituted heteroalkyl group. In some embodiments of Formula II, R₈ and R₉ are hydrogen, R₁₀ and R₁₁ are independently an unsubstituted alkyl group or a substituted alkyl group, for example, a methyl group.

In some embodiments of Formula II, R₁₂ is a hydrogen atom, a hydroxyl group, an alkoxy group, an ether group, a sulfide group, a thiol group, an amino group, or an acyl group optionally containing an unsubstituted alkyl group, a substituted alkyl group, a hydroxyl group, a thiol group, or an amino group. In some embodiments of Formula II, R₁₂ is a hydrogen atom. In some embodiments of Formula II, R₁₂ is a hydroxyl group or an alkoxy group, for example, a methoxy group. In some embodiments of Formula II, R₈-R₁₁ are hydrogen and R₁₂ is a hydroxyl group or an alkoxy group, preferably a methoxy group. R₁₂ can be a substituted or unsubstituted C₁-C₃₀ alkoxy, linear C₁-C₃₀ alkoxy, branched C₃-C₃₀ alkoxy, C₁-C₂₀ alkoxy, linear C₁-C₂₀ alkoxy, branched C₃-C₂₀ alkoxy, C₁-C₁₀ alkoxy, linear C₁-C₁₀ alkoxy, branched C₃-C₁₀ alkoxy, C₁-C₆ alkoxy, linear C₁-C₆ alkoxy, branched C₃-C₆ alkoxy, C₁-C₄ alkoxy, linear C₁-C₄ alkoxy, branched C₃-C₄ alkoxy, C₁-C₃ alkoxy, linear C₁-C₃ alkoxy, branched C₃ alkoxy, linear C₁-C₂ alkoxy, or methoxy.

In some embodiment of Formula II, R₁-R₄, when present, are hydrogen; R₅ is an unsubstituted alkyl group or a substituted alkyl group, for example, a methyl group; R₆, R₇, when present, are independently a hydrogen atom, an unsubstituted alkyl group or a substituted alkyl group, for example, a methyl group; R₈-R₁₁ are independently a hydrogen atom, an unsubstituted alkyl group, or a substituted alkyl group, for example, a methyl group; and R₁₂ is a hydrogen atom, a hydroxyl group, or an alkoxy group, for example, a methoxy group.

In some embodiments of Formula II, each R₁-R₁₂ can independently be a hydrogen atom, an unsubstituted alkyl group, such as an unsubstituted linear alkyl group, an unsubstituted branched alkyl group, an unsubstituted cyclic alkyl group, an unsubstituted linear C₁-C₃₀ alkyl group, an unsubstituted branched C₄-C₃₀ alkyl group, an unsubstituted cyclic C₃-C₃₀ alkyl group, an unsubstituted linear C₁-C₂₀ alkyl group, an unsubstituted branched C₄-C₂₀ alkyl group, an unsubstituted cyclic C₃-C₂₀ alkyl group, an unsubstituted linear C₁-C₁₀ alkyl group, an unsubstituted branched C₄-C₁₀ alkyl group, an unsubstituted cyclic C₃-C₁₀ alkyl group, an unsubstituted linear C₁-C₅ alkyl group, an unsubstituted branched C₄-C₅ alkyl group, an unsubstituted cyclic C₃-C₅ alkyl group, an unsubstituted linear C₁-C₃ alkyl group, an unsubstituted linear C₁-C₂ alkyl group, an unsubstituted cyclic C₃-C₃₀ alkyl group, an unsubstituted cyclic C₃-C₂₀ alkyl group, an unsubstituted cyclic C₃-C₁₀ alkyl group, an unsubstituted cyclic C₃-C₅ alkyl group, an unsubstituted linear C₁-C₃₀ alkyl group, an unsubstituted linear C₁-C₂₀ alkyl group, an unsubstituted linear C₁-C₁₀ alkyl group, an unsubstituted linear C₁-C₅ alkyl group, an unsubstituted linear C₁-C₃ alkyl group, an unsubstituted linear C₁-C₂ alkyl group, or an unsubstituted methyl groups.

In some embodiments of Formula II, each R₁-R₁₂ can independently be a substituted alkyl group, such as a substituted linear alkyl group, a substituted branched alkyl group, or a substituted cyclic alkyl group, a substituted linear C₁-C₃₀ alkyl group, a substituted branched C₃-C₃₀ alkyl group, a substituted cyclic C₃-C₃₀ alkyl group, a substituted linear C₁-C₂₀ alkyl group, a substituted branched C₃-C₂₀ alkyl group, a substituted cyclic C₃-C₂₀ alkyl group, a substituted linear C₁-C₁₀ alkyl group, a branched C₃-C₁₀ alkyl group, a cyclic C₃-C₁₀ alkyl group, a substituted linear C₁-C₅ alkyl group, a substituted branched C₃-C₅ alkyl group, a substituted cyclic C₃-C₅ alkyl group, a substituted linear C₁-C₃ alkyl group, a substituted linear C₁-C₂ alkyl group, a substituted cyclic C₃-C₃₀ alkyl group, a substituted cyclic C₃-C₂₀ alkyl group, a substituted cyclic C₃-C₁₀ alkyl group, a substituted cyclic C₃-C₅ alkyl group, a substituted cyclic C₃ alkyl group, a substituted linear C₁-C₃₀ alkyl group, a substituted linear C₁-C₂₀ alkyl group, a substituted linear C₁-C₁₀ alkyl group, a substituted linear C₁-C₅ alkyl group, a substituted linear C₁-C₃ alkyl group, or a substituted linear C₁-C₂ alkyl group.

In some embodiments of Formula II, P′ is an oxygen atom, a sulfur atom, —NH, or absent; m is zero or a positive integer between 1 and 25, between 1 and 20, between 1 and 15, between 1 and 10, between 1 and 5, or between 1 and 3, such as 1 or 2; Q′ is —CR₆═CR₇—; n is zero or a positive integer between 1 and 25, between 1 and 20, between 1 and 15, between 1 and 10, between 1 and 5, or between 1 and 3, such as 1 or 2; R₁₂ can be a hydroxyl group, an alkoxy group, an ether group, a sulfide group, a thiol group, an amino group, or an acyl group optionally containing an unsubstituted alkyl group, a substituted alkyl group, a hydroxyl group, a thiol group, or an amino group; and each of R₁-R₁₁ is independently hydrogen, an unsubstituted alkyl group, or a substituted alkyl group, such as an unsubstituted C₁-C₂₀ alkyl group, an unsubstituted C₁-C₁₅ alkyl group, an unsubstituted C₁-C₁₂ alkyl group, an unsubstituted C₁-C₁₀ alkyl group, an unsubstituted C₁-C₅ alkyl group, an unsubstituted C₁-C₆ alkyl group, an unsubstituted C₁-C₅ alkyl group, an unsubstituted C₁-C₄ alkyl group, an unsubstituted C₁-C₃ alkyl group, an unsubstituted C₁-C₂ alkyl group, or a methyl group.

In some embodiments, the compound can have the structures of Formula II′:

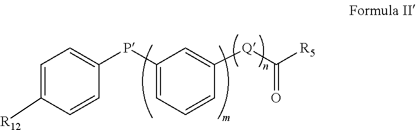

where P′ is an oxygen atom, a sulfur atom, —NH, or absent; m is zero or a positive integer between 1 and 25, between 1 and 20, between 1 and 15, between 1 and 10, between 1 and 5, or between 1 and 3, such as 1 or 2; Q′ is

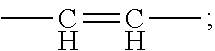

n is zero or a positive integer between 1 and 25, between 1 and 20, between 1 and 15, between 1 and 10, between 1 and 5, or between 1 and 3, such as 1 or 2; R₁₂ can be a hydroxyl group, an alkoxy group, an ether group, a sulfide group, a thiol group, an amino group, or an acyl group optionally containing an unsubstituted alkyl group, a substituted alkyl group, a hydroxyl group, a thiol group, or an amino group; R₅ is an unsubstituted alkyl group or a substituted alkyl group, such as an unsubstituted C₁-C₂₀ alkyl group, an unsubstituted C₁-C₁₅ alkyl group, an unsubstituted C₁-C₁₂ alkyl group, an unsubstituted C₁-C₁₀ alkyl group, an unsubstituted C₁-C₅ alkyl group, an unsubstituted C₁-C₆ alkyl group, an unsubstituted C₁-C₅ alkyl group, an unsubstituted C₁-C₄ alkyl group, an unsubstituted C₁-C₃ alkyl group, an unsubstituted C₁-C₂ alkyl group, or a methyl group.

In some preferred embodiments of Formula II′, the length from P′ to

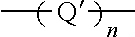

is about the length of a C₆ chain.

In some embodiments of Formula II′, P′ is an oxygen atom or absent; m is a positive integer between 1 and 6, between 1 and 5, between 1 and 4, between 1 and 3, between 1 and 2, or 1; n is 0 or an integer between 1 and 5, between 1 and 4, between 1 and 3, between 1 and 2, or 1; R₁₂ is a hydroxyl group, an alkoxy group, an ether group, a sulfide group, a thiol group, or an amino group, optionally a hydroxyl group or an alkoxy group; and R₅ is an unsubstituted C₁-C₁₀ alkyl group, an unsubstituted C₁-C₈ alkyl group, an unsubstituted C₁-C₆ alkyl group, an unsubstituted C₁-C₅ alkyl group, an unsubstituted C₁-C₄ alkyl group, an unsubstituted C₁-C₃ alkyl group, an unsubstituted C₁-C₂ alkyl group, or a methyl group.

In some embodiments, the compound can have the structures of Formula II″:

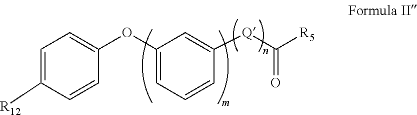

where Q′ is

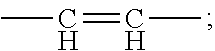

m is a positive integer between 1 and 5, between 1 and 4, between 1 and 3, between 1 and 2, or 1; n is an integer between 1 and 4, between 1 and 3, between 1 and 2, or 1; R₁₂ is a hydroxyl group, an alkoxy group, an ether group, a sulfide group, a thiol group, or an amino group, optionally a hydroxyl group or an alkoxy group; and R₅ is an unsubstituted C₁-C₁₀ alkyl group, an unsubstituted C₁-C₈ alkyl group, an unsubstituted C₁-C₆ alkyl group, an unsubstituted C₁-C₅ alkyl group, an unsubstituted C₁-C₄ alkyl group, an unsubstituted C₁-C₃ alkyl group, an unsubstituted C₁-C₂ alkyl group, or a methyl group. In some preferred embodiments of Formula II″, m is 1 and n is 1.

In some embodiments, the compound can have the structures of Formula II′″:

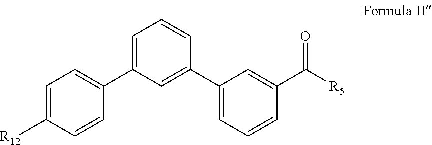

where R₁₂ is a hydroxyl group, an alkoxy group, an ether group, a sulfide group, a thiol group, or an amino group, optionally a hydroxyl group or an alkoxy group; and R₅ is an unsubstituted C₁-C₁₀ alkyl group, an unsubstituted C₁-C₈ alkyl group, an unsubstituted C₁-C₆ alkyl group, an unsubstituted C₁-C₅ alkyl group, an unsubstituted C₁-C₄ alkyl group, an unsubstituted C₁-C₃ alkyl group, an unsubstituted C₁-C₂ alkyl group, or a methyl group.

In some embodiments, the compound can have the structures of Formula III:

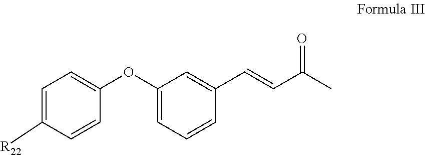

where R₂₂ can be a hydroxyl group, an alkoxy group, an ether group, or a thiol group. Preferably, R₂₂ is an alkoxy group, more preferably, a methoxy group.

In some embodiments, the compound can have the structures of Formula IV:

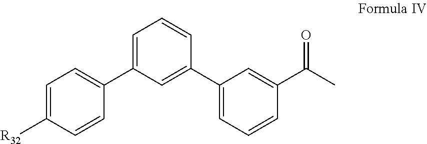

where R₃₂ can be a hydroxyl group, an alkoxy group, an ether group, or a thiol group. Preferably, R₃₂ is an alkoxy group, more preferably, a methoxy group.

Exemplary and preferred compounds having the structure of Formula I or Formula II include compounds MZ1-MZ8, the structure of which are shown below.

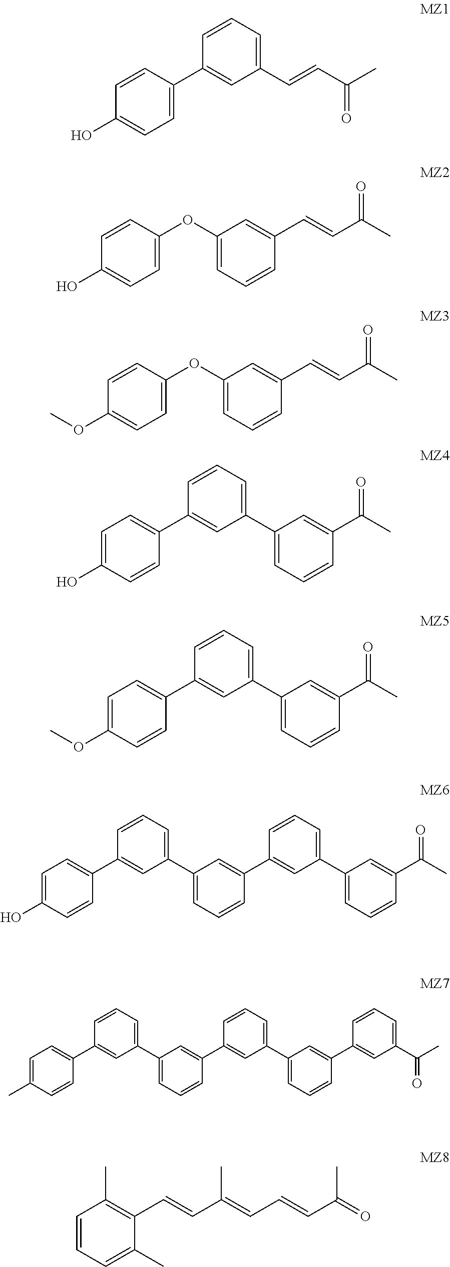

In some embodiments, the salts of Formulas I and II can be prepared by treating the free acid form of the compounds with an appropriate amount of a base. Exemplary bases are ammonium hydroxide, sodium hydroxide, potassium hydroxide, lithium hydroxide, calcium hydroxide, magnesium hydroxide, ferrous hydroxide, zinc hydroxide, copper hydroxide, aluminum hydroxide, ferric hydroxide, isopropylamine, trimethylamine, diethylamine, triethylamine, tripropylamine, ethanolamine, 2-dimethylaminoethanol, 2-diethylaminoethanol, lysine, arginine, histidine, and the like.

B. Formulations

The compounds disclosed herein or salts thereof may be used in unmodified form, but are generally formulated into formulations in combination with one or more formulation excipients, in a suitable carrier. The disclosed compounds are included in the formulation in an effective amount to inhibit SL biosynthesis and/or release in a plant.

The formulations can be in various forms, as described below.

Formulation excipient are generally materials that can be used to deliver active ingredients, such as the compounds described herein, to a plant, a plant part (e.g., a seed or seedling), or a growing site of a plant (e.g., soil or growth medium), without having an adverse effect on plant growth, growth medium structure, soil drainage, or the like.

Generally, the compounds or salts thereof in a formulation is in an effective amount to promote plant growth, and/or inhibit growth of parasitic weed.

The formulations may contain from about 0.1% to about 95% of the compounds or salts thereof by weight, such as between about 5% and about 95%, between about 0.1% and about 90% by weight, between about 1% and about 80% by weight, between about 1% and about 60% by weight, between about 1% and about 50% by weight, between about 1% and about 40% by weight, between about 1% and about 30% by weight, between about 1% and about 20% by weight, or between about 1% and about 10% by weight.

In embodiments where the formulation is a liquid, the compounds or salts thereof can have a concentration between about 0.1 μM and about 10 M, between about 1 μM and about 1 M, between about 1 μM and about 100 mM, or between about 1 μM and about 10 mM, such as up to about 1 M, up to about 500 mM, up to about 100 mM, at least 1 mM, at least 10 mM, or at least 50 mM.

In embodiments where the formulation is diluted prior to use, after dilution, the compounds or salts thereof can have a concentration between about 0.1 μM and about 1 mM, between about 0.1 μM and about 10 μM, or between about 1 μM and about 10 μM, such as about 2.5 μM or about 5 μM.

The specific amount of compounds in the formulation depends on the formulation form, application equipment, and nature of the plants to be treated.

1. Formulation Excipients and Carriers

Suitable formulation excipients are known in the art and are described below. The carrier can be any carrier, including, but not limited to, solid carriers and liquid carriers.

Optionally, more than one suitable formulation excipient may be formulated together and/or mixed separately.

Formulating formulations containing one or more active ingredients, such as compounds disclosed herein, are known in the art.

a. Solid Carriers

Suitable solid carriers include, but are not limited to, plant powders (e.g., soybean flour, tobacco flour, wheat flour, wood flour, walnut shell four, cotton seed hulls, and the like), mineral powders (e.g., clays such as attapulgite clay, kaolin clay, Fubasami clay, pyrophyllite clay, bentonite and acid clay, tales such as talc powder and agalmatolite powder, silicas such as diatomaceous earth and mica powder, pumice, fuller's earth, diatomaxeous earth, and the like), synthetic hydrated silicon oxide, alumina, talc, kieselguhr, chalk, titanium dioxide, ceramic, other inorganic minerals (e.g., sericite, quartz, sulfur, active carbon, calcium carbonate, hydrated silica, lime, and the like), lignin, and a combination thereof.

b. Liquid Carriers

Suitable liquid carriers include, but are not limited to, water, alcohols (e.g., methyl alcohol, ethyl alcohol, n-propyl alcohol, isopropyl alcohol, butyl alcohol, hexyl alcohol, benzyl alcohol, ethylene glycol, propylene glycol, phenoxyethanol), ketones (e.g., acetone, methyl ethyl ketone, methyl isobutyl ketone, cyclohexanone), ethers (e.g., diisopropyl ether, 1,4-dioxane, tetrahydrofuran, ethylene glycol monomethyl ether, ethylene glycol dimethyl ether, diethylene glycol monomethyl ether, propylene glycol monomethyl ether, dipropylene glycol monomethyl ether, 3-methoxy-3-methyl-1-butanol), aliphatic hydrocarbons (e.g., hexane, cyclohexane, kerosene, lamp oil, fuel oil, machine oil and so on), aromatic hydrocarbons (e.g., toluene, xylene, ethylbenzene, dodecylbenzene, phenylxylylethane, solvent naphtha, methylnaphthalene), halogenated hydrocarbons (e.g., dichloromethane, trichloroethane, chloroform, carbon tetrachloride), acid amides (e.g., N,N-dimethylformamide, N,N-dimethylacetamide, N-methylpyrrolidone, N-octylpyrrolidone), esters (e.g., butyl lactate, ethyl acetate, butyl acetate, isopropyl myristate, ethyl oleate, diisopropyl adipate, diisobutyl adipate, propylene glycol monomethyl ether acetate, fatty acid glycerin ester, Y-butyrolactone), nitriles (e.g., acetonitrile, isobutyronitrile, propionitrile), carbonates (e.g., propylene carbonate and so on), and vegetable oils (e.g., soybean oil, olive oil, linseed oil, coconut oil, palm oil, peanut oil, malt oil, almond oil, sesame oil, mineral oil, rosmarinic oil, geranium oil, rapeseed oil, cotton seed oil, corn oil, safflower oil, orange oil).

Water is generally the carrier of choice for the dilution of concentrates.

In some embodiments, the liquid carrier is water, acetone, cyclohexanone, or a combination thereof.

c. Surface-Active Agents

Surface-active agents may be mixed with any solid and liquid carriers described above to form the formulations. Optionally, one or more surface-active agents are included in formulations designed to be diluted with a carrier, such as water, before application.

Surface-active agents can be anionic, cationic, non-ionic or polymeric.

Optionally, surface-active agents are employed as emulsifying agents, wetting agents, and/or suspending agents.

Suitable surface-active agents include, but are not limited to, salts of alkyl sulfates, such as Atlas G-1086 (product name, manufactured by Croda industrial chemicals), diethanolammonium lauryl sulphate; alkylarylsulfonate salts, such as calcium dodecylbenzenesulfonate; alkylphenol-alkylene oxide addition products, such as nonylpheno 1-C. sub. 18 ethoxylate; alcohol-alkylene oxide addition products, such as tridecyl alcoho 1-C. sub. 16 ethoxylate; soaps, such as sodium stearate; alkylnaphthalenesulfonate salts, such as sodium dibutylnaphthalenesulfonate; dialkyl esters of sulfosuccinate salts, such as sodium di(2-ethylhexyl) sulfosuccinate; sorbitol esters, such as sorbitol oleate; quaternary amines, such as lauryl trimethylammonium chloride; polyethylene glycol esters of fatty acids, such as polyethylene glycol stearate; block copolymers of ethylene oxide and propylene oxide; and salts of mono and dialkyl phosphate esters.

In some embodiments, the surface-active agent is Atlas G-1086.

d. Other Formulation Excipients

Other excipients commonly utilized in agricultural compositions can be included in the disclosed formulations and they include, but are not limited to, crystallisation inhibitors, viscosity modifiers, suspending agents, spray droplet modifiers, pigments, antioxidants, foaming agents, light-blocking agents, compatibilizing agents, antifoam agents, sequestering agents, neutralising agents and buffers, corrosion inhibitors, dyes, odorants, spreading agents, penetration aids, micronutrients, emollients, lubricants, sticking agents, and the like. Casein, gelatin, saccharides (e.g., starch, Xanthan gum, gum arabic, cellulose derivatives, and alginic acid), lignin derivatives, bentonite, synthetic water-soluble polymers (e.g., polyvinyl alcohol, polyvinyl pyrrolidone, polyacrylic acids), PAP (acidic isopropyl phosphate), BHT (2,6-di-tert-butyl-4-methylphenol), and BHA (mixture of 2-tert-butyl-4-methoxyphenol and 3-tert-butyl-4-methoxyphenol) can also be used.

Optionally, the formulations can include one or more fertilizers, for example, liquid fertilizers or solid, particulate fertiliser carriers such as ammonium sulfate, ammonium phosphate, ammonium nitrate, urea, and ammonium chloride.

Optionally, the formulation can include one or more pesticides, such as such as insecticides, nematicides, fungicides or herbicides, or additional plant growth regulators.

Alternatively, the fertilizers, pesticides, and/or additional plant growth regulators can be separate from the formulation and applied concurrently or sequentially with the formulation to a plant or growing site of a plant.

2. Forms of Formulations

The formulations can be in various physical forms, for example, dusting powders, aerosols, gels, wettable powders, water-dispersible granules, water-dispersible tablets, effervescent compressed tablets, emulsifiable concentrates, microemulsifiable concentrates, oil-in-water emulsions, oil flowables, aqueous dispersions, oil dispersions, suspoemulsions capsule suspensions, emulsifiable granules, soluble liquids, water-soluble concentrates (with water or a water miscible organic solvent as carrier), or impregnated polymer films.

The above described formulations can be applied directly or diluted prior to use. Diluted formulation can be prepared, for example, with water.

a. Exemplary Formulations

i. Wettable Powders

Wettable powders are generally in the form of finely divided particles which disperse readily in water or other liquid carriers. The particles contain the compounds retained in a solid matrix.

Suitable solid matrices include, but are not limited to, fuller's earth, kaolin clays, silicas and other readily wet organic or inorganic solids.

Wettable powders normally contain a small amount of surface-active agents as emulsifying agents, wetting agents, and/or suspending agents.

ii. Emulsifiable Concentrates

Emulsifiable concentrates are homogeneous liquid compositions dispersible in water or other liquid and may consist entirely of the active compound with a liquid or solid emulsifying agent, or may also contain a liquid carrier, such as xylene, heavy aromatic naphthas, isophorone and other non-volatile organic solvents.

Emulsifiable concentrates are typically dispersed in water or other liquid carriers as described above.

iii. Granular Formulations

Granular formulations include both extrudates and relatively coarse particles. Granular formulations are generally applied without dilution to a plant or growing site of a plant.

Typical carriers for granular formulations include, but are not limited to, fertilizer, sand, fuller's earth, attapulgite clay, bentonite clays, montmorillonite clay, vermiculite, perlite, calcium carbonate, brick, pumice, pyrophyllite, kaolin, dolomite, plaster, wood flour, ground corn cobs, ground peanut hulls, sugars, sodium chloride, sodium sulphate, sodium silicate, sodium borate, magnesia, mica, iron oxide, zinc oxide, titanium oxide, antimony oxide, cryolite, gypsum, diatomaceous earth, calcium sulphate and other organic or inorganic materials which absorb or can be coated with the active compound.

Granular formulations normally contain one or more surface-active agents such as heavy aromatic naphthas, kerosene and other petroleum fractions, or vegetable oils; and/or stickers such as dextrins, glue, or synthetic resins. The granular substrate material can be a solid carriers described above and/or a fertilizer, such as urea/formaldehyde fertilisers, ammonium, liquid nitrogen, urea, potassium chloride, ammonium compounds, phosphorus compounds, sulphur, similar plant nutrients and micro nutrients and mixtures or combinations thereof.

The compounds may be homogeneously distributed throughout the granule or may be spray impregnated or absorbed onto the granule substrate after the granules are formed.

iv. Encapsulated Granules

Encapsulated granules are generally porous granules with porous membranes sealing the granule pore openings, retaining the compounds in liquid form inside the granule pores.

Granules typically have a diameter ranging from about 1 mm to about 1 cm, such as from about 1 mm to about 50 mm, from about 1 mm to about 20 mm, from about 1 mm to about 10 mm, from about 1 mm to about 2 mm.

Granules are formed by extrusion, agglomeration or prilling, or are naturally occurring. Examples of naturally occurring granule materials include, but are not limited to, vermiculite, sintered clay, kaolin, attapulgite clay, sawdust, and granular carbon. Exemplary materials for porous membranes include, but are not limited to, natural and synthetic rubbers, cellulosic materials, styrene-butadiene copolymers, polyacrylonitriles, polyacrylates, polyesters, polyamides, polyureas, polyurethanes, and starch xanthates.

v. Dusts

Dusts are free-flowing admixtures of the compounds with finely divided solids, such as talc, clays, flours and other organic and inorganic solids. The finely divided solids act as dispersants and carriers.

vi. Microcapsules

Microcapsules are typically droplets or granules where active compounds enclosed in an inert porous shell, which allows escape of the enclosed compounds to the surroundings at controlled rates.

vii. Encapsulated Droplets

Encapsulated droplets are typically about 1 to 50 microns in diameter. The enclosed liquid may include a liquid carrier as described above in addition to the compounds.

b. Other Formulations

Other useful formulations include simple solutions of the compounds in a solvent in which it is completely soluble at the desired concentration, such as acetone, cyclohexanone, alkylated naphthalenes, xylene, and other organic solvents.

Pressurised sprayers, wherein the compounds are dispersed in finely-divided form as a result of vaporisation of a low boiling dispersant solvent carrier, may also be used.

### II. Methods of Making the Compounds

Disclosed are methods of making the compounds.

The synthetic path of MZ is simple (only one or two steps), which hugely lower the cost and chemical waste. The synthesis produces the compounds with high yield. The synthetic method for the compounds is significantly more effective than the synthesis of zaxinone, which typically takes 4-5 steps with relative low yield.

An exemplary method for making the compounds of Formula I or Formula II is described in Example 1.

### III. Methods of Using the Compounds

Methods of using the compounds, or salts thereof, and formulations of the compounds or salts thereof are disclosed.

The compounds or salts thereof may be directly applied to a plant, a plant part, or a growing site of plant in an unmodified form. Preferably, a formulation of the compounds or salts thereof is applied to the plant, the plant part, or the growing site of plant. Any formulations described above may be employed.

The compounds are the first described synthetic compounds with Zaxinone (Zax) activity. In some embodiments, the compounds show high efficiency in accelerating plant growth, combating root parasitic weeds (e.g., Striga), and regulating plant architecture (i.e., number of branches, root type, and root growth), which, in some applications, exceeds that of natural plant metabolites, such as Zaxinone (Zax). For example, the compounds disclosed herein can promote root growth of a wild-type plant seedling by increasing root length, number of crown roots, root biomass, and/or tiller number, and/or rescue root-related plant mutant (e.g. increasing root length, number of crown roots, root biomass, and/or tiller number). In some embodiments, plant treated with the compounds disclosed herein can have an increased height, increased leave number and/or individual leave area, increased braches, increased flower number, increased root length, increased root fresh weight, and/or increased fresh weight, and/or can produce a higher number of fruit, fruit with improved fruit quality (e.g. increased fruit weight, fruit size, firmness, and acidity, lower dissolfided solids, increased vitamin C content and phenol content, etc.), and/or with an increased yield compared to the same but untreated plant, such as green pepper plant.

While not being bound by theory, the compounds' ability to promote plant growth and combat root parasitic weeds are by their activity of suppressing transcript level of SL biosynthesis genes in a host plant. SLs have been identified to be responsible for three different physiological processes: (1) they promote the germination of parasitic organisms that grow in the host plant's roots, such as Striga and other plants of the genus Striga; (2) SLs are fundamental for the recognition of the plant by symbiotic fungi, especially arbuscular mycorrhizal fungi, because they establish a mutualistic association with these plants, and provide phosphate and other soil nutrients; and (3) SLs have been identified as branching inhibition hormones in plants; when present, these compounds prevent excess bud growing in stem terminals, stopping the branching mechanism in plants (Umehara, et al., Plant & Cell Physiology, 56(6):1059-1072 (2015)). The compounds disclosed herein can suppress SL biosynthesis in a host plant and its release from the host plant, and thereby promote growth of the host plant (e.g. corp) and combat root parasitic weeds (e.g. Striga).

A. Methods for Promoting Plant Growth

Generally, a method for promoting plant growth includes (i) applying one or more formulations described above to a plant, plant part, or growing site of plant.

Step (i) may be repeated, (e.g., more than once). Optionally, step (i) may be repeated twice, three times, four times, five times, six times, seven times, eight times, nine times, ten times, etc. Optionally, step (i) may be repeated once every two days, once every three days, once every week, once every two weeks, once every month, once every two months, once every three months, once every six month, once every year, etc.

Optionally, step (i) may be repeatedly applied to different objects of a plant and/or growing site of the plant at each time. For example, the one or more formulations may be applied to a seed of a plant, seedling of the plant, a growing site (e.g. soil or a growing medium) before, during, or after planting of the plant, and/or to the root of the plant.

1. Plants

a. Plant Types

In some embodiments, the plant to be treated can be a cereal, grain, or vegetable plant. The plant may be grown in soil or in a growing medium (i.e. hydroponically grown plant). Exemplary plants include, but are not limited to, corn, rice, maize, wheat, barley, rye, oat, sorghum, pearl millet, millet, cotton, bean, soybean, pea, peanut, pepper, buckwheat, beet, rapeseed, sunflower, sugarcane, tobacco, solanaceous vegetables (eggplant, tomato, pimento, pepper, potato, etc.), hemp, clover, melon, legume, cucurbitaceous vegetables (cucumber, pumpkin, zucchini, water melon, melon, squash, etc.), cruciferous vegetables (Japanese radish, white turnip, horseradish, kohlrabi, Chinese cabbage, cabbage, leaf mustard, broccoli, cauliflower, etc.), asteraceous vegetables (burdock, crown daisy, artichoke, lettuce, etc.), liliaceous vegetables (green onion, onion, garlic, and asparagus), ammiaceous vegetables (carrot, parsley, celery, parsnip, etc.), chenopodiaceous vegetables (spinach, Swiss chard, etc.), lamiaceous vegetables (Perilla frutescens, mint, basil, etc.), strawberry, potato, sweet potato, Dioscorea japonica, colocasia, etc.

In some preferred embodiments, the plant is a rice plant, a pepper plant, a strawberry plant, or a potato plant, such as a rice plant or a pepper plant (e.g. green pepper).

The plant may be wild-type or mutant. In some embodiments, the plant is a wild-type plant, such as a wild-type rice plant, a wild-type pepper plant (e.g. green pepper), a wild-type strawberry plant, or a wild-type potato plant.

In some embodiments, the plant is a mutant, such as a zas mutant (i.e. a Zaxinone deficient mutant). For example, the plant is a zas rice mutant, a zas pepper mutant (e.g. green pepper), a zas strawberry mutant, or a zas potato mutant. In some embodiments, the one or more formulations, which are applied to the plant, plant part, or growing site of plant, contain an effective amount of the compounds or salts thereof to rescue a plant mutant. For example, the one or more formulations applied to the plant, plant part, or growing site of the plant contain an effective amount of the compounds or salts thereof to rescue growth retardation of the plant mutant, such as a zas rice mutant, a zas pepper mutant, a zas strawberry mutant, or a zas potato mutant.

The formulations can be applied to seedlings, mature plants or plant parts. Exemplary plant parts that can be treated include foliages, seeds, roots, and bulbs. Bulb generally refers to a bulb, corm, rhizoma, stem tuber, root tuber, and rhizophore.

In some embodiments, the plant part is seed or seedling.

b. Growing Sites

The growing site of plant can be soil or a growing medium (e.g. hydroponically grown plant). The soil or growing medium may be filled in a suitable container for planting the plant, such as a pot or a Rhizotron system. The growing site may be under greenhouse conditions or in the field.

In some embodiments, the growing site of plant is soil. The soil may be soil before, during, or after planting the plant.

In some embodiments, the growing site of plant is a growing medium. Exemplary growing medium include, but are not limited to, water, culture solution, urethane, and rock wool. Culture solution generally refers to a water solution containing nutrient components required for plant growth. The nutrient components and their relative concentrations can be easily adjusted to a proper concentration for different plants and application, which are known in the art.

2. Applying the Formulations

The one or more formulations may be applied to a plant, plant part, or growing site of plant by any method known in the art, including both foliar and non-foliar application.

Exemplary application method to a plant or plant part include, but are not limited to, spraying, drenching, dripping on, or dusting the plant or plant part, coating a seed, and/or applying as a cream or paste or as a vapor.

For example, application methods for a foliage of plants may be applying to surfaces of plants, such as foliage spraying and trunk spraying. In some embodiments, the method of application can be absorbing to plants transplantation such as soaking entire plant or roots. In some embodiments, a formulation formulated with a solid carrier may be adhered to the roots.

Methods for coating a seed is known in the art, for example, US 2007/0105721 by Flematti, et al.

Exemplary application methods to soil include, but are not limited to, spraying onto the soil, drenching the soil, dripping onto the soil, dusting the soil, and/or soil incorporation.

Examples of places where the one or more formulation can be applied include, but are not limited to, planting hole, furrow, around a planting hole, around a furrow, entire surface of cultivation lands, the parts between the soil and the plant, area between roots, area beneath the trunk, main furrow, growing soil, seedling raising box, seedling raising tray and seedbed.

Examples of the treating period include before seeding, at the time of seeding, immediately after seeding, raising period, before settled planting, at the time of settled planting, and growing period after settled planting.

Exemplary application method to a growing medium, such as a culture solution, include, but are not limited to, mixing the compounds or formulations into the growing medium. The growing medium containing the compounds or formulations may be applied, for example, to soak a plant or plant part, such as seeds or seedling for their germination or rotting, or to soak roots of plants or spraying it to roots to culture the plants.

3. Optional Steps

a. Diluting the Formulations

Optionally, the method can include a step of diluting the formulation in a liquid carrier prior to step (i). The liquid carrier can be any liquid carrier described above, such as water.

b. Applying Agriculturally Beneficial Agents

Optionally, the method can include a step of applying one or more agriculturally beneficial agents prior to, during, or after step (i). Agriculturally beneficial agents generally refer to any agent or combination of agents capable of causing or providing a beneficial and/or useful effect in agriculture.

In some embodiments, the one or more agriculturally beneficial agents are applied prior to applying the one or more formulations of the compounds or salts thereof.

In some embodiments, the one or more agriculturally beneficial agents are applied during (e.g., simultaneous or substantially simultaneous with) applying the one or more formulations of the compounds or salts thereof.

In some embodiments, the one or more agriculturally beneficial agents are applied after applying the one or more formulations of the compounds or salts thereof.

Optionally, the agriculturally beneficial agents may be part of a formulation described above, or independently from the one or more formulations.

The agriculturally beneficial agents can be fertilizers, micronutrients, microorganisms, or a combination thereof.

Fertilizers are generally known to the person skilled in the art, e.g., see Ullmann's Encyclopedia of Industrial Chemistry, 5^{th} edition, Vol. A 10, Verlagsgesellschaft, Weinheim, 1987. Exemplary fertilizers that can be used in the disclosed methods are generally organic and inorganic nitrogen-containing compounds, such as ureas, urea/formaldehyde condensates, amino acids, ammonium salts and ammonium nitrates, potassium salts (e.g., chlorides sulphates, nitrates), salts of phosphoric acid, salts of phosphorous acid (e.g., potassium salts and ammonium salts), and a combination thereof.

Exemplary micronutrients include, but are not limited to calcium, sulphur, boron, manganese, magnesium, iron, copper, zinc, molybdenum, cobalt, and a combination thereof.

Exemplary microorganisms include, but are not limited to nitrogen fixing microorganisms, phosphate solubilizing microorganisms, mycorrhizal fungi, and a combination thereof.

c. Planting a Plant or Plant Part

The method may include a step of planting a plant or plant part. The planting step may occur before, during, or after step (i).

In some embodiments, a plant or plant part is planted prior to applying the one or more formulations of the compounds or salts thereof to the plant, plant part, or growing site of the plant.

In some embodiments, the plant or plant part is planted during (e.g., simultaneous or substantially simultaneous with) applying the one or more formulations of the compounds or salts thereof to the plant, plant part, or growing site of the plant.

In some embodiments, the plant or plant part is planted after applying the one or more formulations of the compounds or salts thereof to the plant, plant part, or growing site of the plant.

4. Effective Amount

Generally, for promoting plant growth, the one or more formulations can contain an effective amount of compounds or salts thereof from 0.1% to about 95% of the compounds or salts thereof by weight, such as between about 5% and about 95%, between about 0.1% and about 90% by weight, between about 1% and about 80% by weight, between about 1% and about 60% by weight, between about 1% and about 50% by weight, between about 1% and about 40% by weight, between about 1% and about 30% by weight, between about 1% and about 20% by weight, or between about 1% and about 10% by weight.

Alternatively, the one or more formulations can contain a concentration of compounds or salts thereof between about 50 nM and about 10 M, between about 0.1 μM and about 10 M, between about 50 nM and about 1 M, between about 0.1 μM and about 1 M, between about 1 μM and about 1 M, between about 1 μM and about 100 mM, or between about 1 μM and about 10 mM, such as up to about 1 M, up to about 500 mM, up to about 100 mM, at least 50 nM, at least 0.1 μM, at least 1 μM, at least 10 μM, at least 1 mM, at least 10 mM, or at least 50 mM.

The term “effective amount” generally refers to the amount, concentration, or dosage of the one or more compounds or salts thereof sufficient to cause the desired results.

In some embodiments, the one or more formulations are diluted before applying to a plant, plant part, or growing site of plant. In such cases, the concentration of the one or more compounds or salts thereof in the diluted formulation is between about 50 nM and about 1 mM, between about 0.1 μM and about 1 mM, between about 0.1 μM and about 100 μM, between about 1 μM and about 100 μM, between about 0.1 μM and about 100 μM, or between about 1 μM and about 10 μM, such as about 2.5 μM, about 5 μM, or about 10 μM.

In some embodiments, the one or more formulations contain an effective amount of the compounds or salts thereof to decrease biosynthesis and release of SL from the host plant. Optionally, the biosynthesis and release of strigolactone plant hormones from the plant is decreased by at least about 40% compared to an untreated plant under same conditions (e.g. growing site, temperature, humidity, pressure, etc.).

In some embodiments, the one or more formulations contain an effective amount of the compounds or salts thereof to increase root length, crop root number, root biomass, and/or tiller number of a host plant compared to the same untreated plant, such as a wild-type rice plant, a wild-type pepper plant (e.g. green pepper), a wild-type strawberry plant, a wild-type potato plant, a zas rice mutant, a zas pepper mutant (e.g. green pepper), a zas strawberry mutant, or a zas potato mutant, where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, the one or more formulations contain an effective amount of the compounds or salts thereof to increase root length of a host plant by at least about 10%, at least 15%, at least about 20%, at least 25%, at least about 30%, at least 35%, at least about 40%, at least 45%, at least about 50%, at least 55%, at least about 60%, at least 65%, at least about 70%, at least 75%, at least about 80%, at least about 85%, at least about 90%, or at least about 100% compared to a control (the same plant not treated with the compounds or salts thereof), optionally where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, the one or more formulations contain an effective amount of the compounds or salts thereof to increase the crown root number of a host plant by at least about 10%, at least 15%, at least about 20%, at least 25%, at least about 30%, at least 35%, at least about 40%, at least 45%, at least about 50%, at least 55%, at least about 60%, at least 65%, at least about 70%, at least 75%, at least about 80%, at least about 85%, at least about 90%, or at least about 100% compared to a control, optionally where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, the one or more formulations contain an effective amount of the compounds or salts thereof to increase root biomass of a host plant by at least about 10%, at least 15%, at least about 20%, at least 25%, at least about 30%, at least 35%, at least about 40%, at least 45%, at least about 50%, at least 55%, at least about 60%, at least 65%, at least about 70%, at least 75%, at least about 80%, at least about 85%, at least about 90%, or at least about 100% compared to a control, optionally where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, the one or more formulations contain an effective amount of the compounds or salts thereof to increase tiller number of a host plant by at least about 10%, at least 15%, at least about 20%, at least 25%, at least about 30%, at least 35%, at least about 40%, at least 45%, at least about 50%, at least 55%, at least about 60%, at least 65%, at least about 70%, at least 75%, at least about 80%, at least about 85%, at least about 90%, or at least about 100% compared to a control, optionally where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, the one or more formulations contain an effective amount of the compounds or salts thereof to increase root length, crop root number, root biomass, and tiller number of a host plant and each of the root length, crop root number, root biomass, and tiller number of the host plant is increased by at least about 10%, at least about 20%, at least about 30%, at least about 40%, at least about 50%, at least about 60%, at least about 70%, at least about 80%, at least about 90%, or at least about 100% compared to a control, optionally where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, plant treated with the one or more formulations containing an effective amount of the compounds or salts thereof can have an increased height, increased leave number and/or individual leave area, increased branches, increased root length, increased root fresh weight, increased flower number, and/or increased plant fresh weight, and/or can produce an increased number of fruit, increased root fresh weight; alternatively or additionally, the treated plant can produce a higher number of fruit, fruit with improved fruit quality (e.g. higher fruit weight, fruit size, firmness, and acidity, increased vitamin C content and phenol content, etc.), and/or with an increased yield compared to the same but untreated plant, such as a green pepper plant, a strawberry plant, or a potato plant, where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, the height, leave number, individual leave area, branch number, root length, root fresh weight, flower number, and/or plant fresh weight of a plant treated with the one or more formulations contain an effective amount of the compounds or salts thereof can increase by at least about 10%, at least 15%, at least about 20%, at least 25%, at least about 30%, at least 35%, at least about 40%, at least 45%, at least about 50%, at least 55%, at least about 60%, at least 65%, at least about 70%, at least 75%, at least about 80%, at least about 85%, at least about 90%, or at least about 100% compared to the same but untreated plant, optionally where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, the plant treated with the one or more formulations contain an effective amount of the compounds or salts thereof can produce an increased number of fruit, fruit with improved quality (e.g. higher fruit weight, fruit size, firmness, and acidity, increased vitamin C content and phenol content, etc.), and/or with an increased yield, where at least one of the number of fruit, fruit quality, and yield is increased by at least about 10%, at least 15%, at least about 20%, at least 25%, at least about 30%, at least 35%, at least about 40%, at least 45%, at least about 50%, at least 55%, at least about 60%, at least 65%, at least about 70%, at least 75%, at least about 80%, at least about 85%, at least about 90%, or at least about 100% compared to the same but untreated plant, optionally where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, the height, leave number, individual leave area, branch number, root length, root fresh weight, flower number, and/or plant fresh weight of a plant treated with the one or more formulations contain an effective amount of the compounds or salts thereof is increased by at least about 10%, at least 15%, at least about 20%, at least 25%, at least about 30%, at least 35%, at least about 40%, at least 45%, at least about 50%, at least 55%, at least about 60%, at least 65%, at least about 70%, at least 75%, at least about 80%, at least about 85%, at least about 90%, or at least about 100% compared to the same but untreated plant and the treated plant also produces an increased number of fruit, fruit with improved quality (e.g. higher fruit weight, fruit size, firmness, and acidity, increased vitamin C content and phenol content, etc.), and/or with an increased yield and at least one of the number of fruit, fruit quality, and yield is increased by at least about 10%, at least 15%, at least about 20%, at least 25%, at least about 30%, at least 35%, at least about 40%, at least 45%, at least about 50%, at least 55%, at least about 60%, at least 65%, at least about 70%, at least 75%, at least about 80%, at least about 85%, at least about 90%, or at least about 100% compared to the untreated plant, optionally where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

B. Methods for Inhibiting Growth of Parasitic Weed

Generally, a method for inhibiting growth of a parasitic weed includes (i) applying one or more formulations described above to the parasitic weed, a part of the parasitic weed, or a growing site of parasitic weed.

Step (i) may be repeated, (e.g., more than once). Optionally, step (i) may be repeated twice, three times, four times, five times, six times, seven times, eight times, nine times, ten times, etc. Optionally, step (i) may be repeated once every two days, once every three days, once every week, once every two weeks, once every month, once every two months, once every three months, once every six month, once every year, etc.

Optionally, step (i) may be repeatedly applied to different objects of a parasitic weed and/or growing site of the parasitic weed at each time. For example, the one or more formulations may be applied to a seed of a parasitic weed, seedling of the parasitic weed, the soil before, during, or after planting of a host plant, and/or to the root of the parasitic weed.

1. Parasitic Weed

Parasitic weed is known in the art.

In some embodiments, the parasitic weed is a Striga species or an Orobanche species of the Orobanchaceae family. In some embodiments, the parasitic weed is a Striga species, such as S. hermonthica. In some embodiments, the root parasitic plant is a B. aegyptiaca species, such as P. aegyptiaca.

The root parasitic plant being treated may be a seedling or a mature plant. A part of the parasitic weed may be foliages, seeds, bulbs, and seedlings of the parasitic weed.

A growing site of parasitic weed may be soil or a growing medium. In some embodiments, the growing site of parasitic weed is soil before, during, or after planting a host plant. A host plant is generally the desired plant to grow, such as a rice plant, a pepper plant (e.g. green pepper), a strawberry plant, or a potato plant. In some embodiments, the one or more formulations are applied to the soil before, during, and/or after planting a host plant, such as a rice plant, a pepper plant, a strawberry plant, or a potato plant.

2. Applying the Formulations

The one or more formulations may be applied directly to a parasitic weed, a part of parasitic weed, or a growing site of parasitic weed using any application methods described above.

3. Optional Steps

a. Diluting the Formulations

Optionally, the method can include a step of diluting the formulation in a liquid carrier prior to step (i). The liquid carrier can be any liquid carrier described above, such as water.

b. Planting a Parasitic Weed or Part of Parasitic Weed

The method may include a step of planting a parasitic weed or a part of parasitic weed. The planting step may occur before, during, or after step (i).

In some embodiments, a parasitic weed or a part of parasitic weed is planted prior to applying the one or more formulations of the compounds or salts thereof to the parasitic weed, part of the parasitic weed, or growing site of the parasitic weed.

In some embodiments, the parasitic weed or a part of parasitic weed is planted during (e.g., simultaneous or substantially simultaneous with) applying the one or more formulations of the compounds or salts thereof to the parasitic weed, part of the parasitic weed, or growing site of the parasitic weed.

In some embodiments, the parasitic weed or a part of parasitic weed is planted after applying the one or more formulations of the compounds or salts thereof to the parasitic weed, part of the parasitic weed, or growing site of the parasitic weed.

c. Applying Herbicides.

The method may include a step of applying one or more herbicides before, during, or after step (i).

In some embodiments, one or more herbicides are applied prior to applying the one or more formulations of the compounds or salts thereof.

In some embodiments, the one or more herbicides are applied during (e.g., simultaneous or substantially simultaneous with) applying the one or more formulations of the compounds or salts thereof.

In some embodiments, the one or more herbicides are applied after applying the one or more formulations of the compounds or salts thereof.

Optionally, the herbicides may be part of a formulation described above, or independently from the one or more formulations.

4. Effective Amount

Generally, for inhibiting growth of parasitic weed, the one or more formulations can contain an effective amount of compounds or salts thereof from 0.1% to about 95% of the compounds or salts thereof by weight, such as between about 5% and about 95%, between about 0.1% and about 90% by weight, between about 1% and about 80% by weight, between about 1% and about 60% by weight, between about 1% and about 50% by weight, between about 1% and about 40% by weight, between about 1% and about 30% by weight, between about 1% and about 20% by weight, or between about 1% and about 10% by weight.

Alternatively, the one or more formulations can contain a concentration of compounds or salts thereof between about 0.1 μM and about 10 M, between about 1 μM and about 1 M, between about 1 μM and about 100 mM, or between about 1 μM and about 10 mM, such as up to about 1 M, up to about 500 mM, up to about 100 mM, at least 1 mM, at least 10 mM, or at least 50 mM.

In some embodiments, the one or more formulations are diluted before applying to a parasitic weed, part of parasitic weed, or growing site of parasitic weed. In such cases, the concentration of the one or more compounds or salts thereof in the diluted formulation is between about 0.1 μM and about 1 mM, between about 0.1 μM and about 10 μM, or between about 1 μM and about 10 μM, such as about 2.5 μM or about 5 μM.

In some embodiments, the one or more formulations contain an effective amount of the compounds or salts thereof to inhibit germination of a seed of the parasitic weed. In some embodiments, the one or more formulations for treating a host plant contain an effective amount of the compounds or salts thereof to reduce the number of root parasitic weed emergence at a growing site of the treated host plant compared to the same growing site with the same but untreated host plant, such as a wild-type rice plant, a wild-type pepper plant, a wild-type strawberry plant, a wild-type potato plant, a zas rice mutant, a zas pepper mutant, a zas strawberry mutant, or a zas potato mutant, where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

In some embodiments, the one or more formulations for treating a host plant contain an effective amount of the compounds or salts thereof to reduce the number of emerging parasitic weed at a growing site of the treated host plant by at least about 10%, at least 15%, at least about 20%, at least 25%, at least about 30%, at least 35%, at least about 40%, at least 45%, at least about 50%, at least 55%, at least about 60%, at least 65%, at least about 70%, at least 75%, at least about 80%, at least about 85%, at least about 90%, or at least about 100% compared to the same growing site with the same but untreated host plant, optionally where the concentration of the compounds or salts thereof in the formulation or the diluted formulation can be in any of the above-described ranges, such as between about 0.1 μM and about 100 μM, between about 1 μM and about 100 μM, between about 0.1 μM and about 10 μM, or between about 1 μM and about 10 μM.

The disclosed compounds and methods can be further understood through the following numbered paragraphs.

1. A compound having a structure of Formula I:

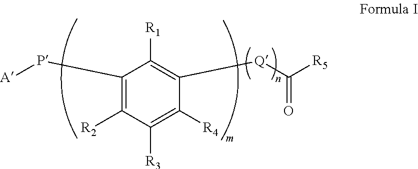

(a) wherein A′ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group;

(b) wherein P′ is an oxygen atom, a sulfur atom, or absent;

(c) wherein m is zero or a positive integer;

(d) wherein Q′ is —CR₆═CR₇—;

(e) wherein n is zero or a positive integer; and

(f) wherein R₁-R₇, when present, are independently

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,

an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonyl group containing one substituent, a carbonate ester group containing one substituent, an ether group containing one substituent, an aminooxy group optionally containing one or two substituents at the amino nitrogen, or a hydroxyamino group optionally containing one or two substituents,

wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

2. The compound of paragraph 1, wherein A′ is an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group.
3. The compound of paragraph 1 or paragraph 2 having a structure of Formula II:

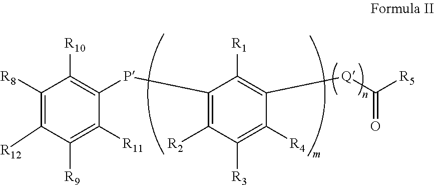

(a) wherein P′, Q′, R₁-R₇, m, and n are as defined in the base paragraphs; and

(b) wherein R₈-R₁₂ are independently

a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,

an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonyl group containing one substituent, a carbonate ester group containing one substituent, an ether group containing one substituent, an aminooxy group optionally containing one or two substituents at the amino nitrogen, or a hydroxyamino group optionally containing one or two substituents,

wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

4. The compound of paragraph 3, wherein R₁₂ is a hydrogen atom, an aldehyde group, an alkoxy group,

an amino group optionally containing one or two substituents at the amino nitrogen, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, an ether group containing one substituent, an aminooxy group optionally containing one or two substituents at the amino nitrogen, or a hydroxyamino group optionally containing one or two substituents,

wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

5. The compound of paragraph 3 or paragraph 4, wherein R₁₂ is a hydrogen atom, a hydroxyl group, an alkoxy group, an ether group, or a thiol group.
6. The compound of any one of paragraphs 1-5, wherein P′ is an oxygen atom.
7. The compound of paragraph 6, wherein the m and n are individually positive integers.
8. The compound of paragraph 6 or paragraph 7, wherein m is 1 and n is 1.
9. The compound of any one of paragraphs 1-5, wherein P′ is absent.
10. The compound of paragraph 9, wherein m is a positive integer and n is zero or 1.
11. The compound of paragraph 9, wherein m is zero and n is a positive integer.
12. The compound of any one of paragraphs 1-11, wherein R₁-R₇, when present, are independently a hydrogen atom, an unsubstituted alkyl group, a substituted alkyl group, an unsubstituted heteroalkyl group, or a substituted heteroalkyl group.
13. The compound of any one of paragraphs 1-12, wherein R₁-R₄ are hydrogen and R₅ is an unsubstituted alkyl group, a substituted alkyl group, an unsubstituted heteroalkyl group, or a substituted heteroalkyl group.
14. The compound of any one of paragraphs 3-13, wherein R₈-R₁₁ are independently a hydrogen atom, an unsubstituted alkyl group, a substituted alkyl group, an unsubstituted heteroalkyl group, or a substituted heteroalkyl group.
15. The compound of any one of paragraphs 3-14, wherein R₈ and R₉ are hydrogen and R₁₀-R₁₁ are independently an unsubstituted alkyl group, a substituted alkyl group, an unsubstituted heteroalkyl group, or a substituted heteroalkyl group.
16. A compound having a structure of:

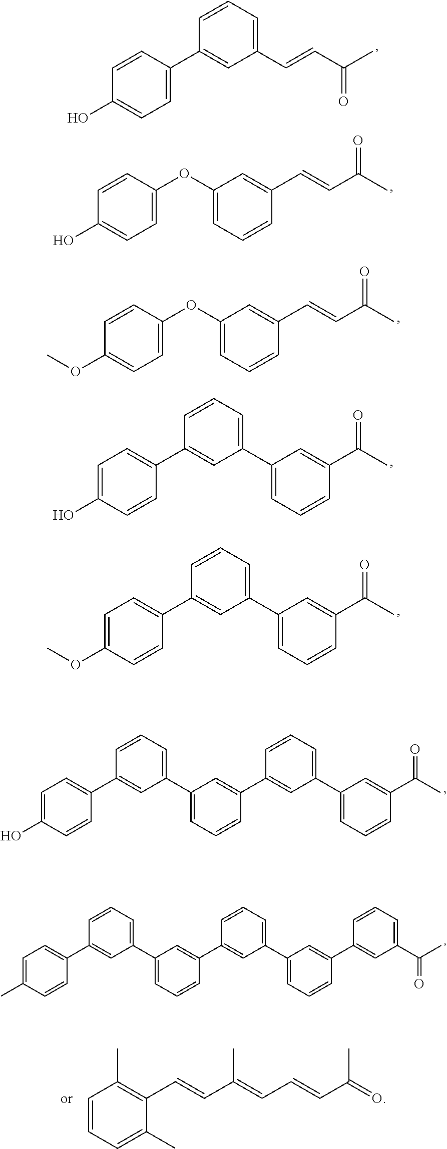

17. A formulation comprising

(a) one or more compounds of any one of paragraphs 1-16, or salts thereof, and

(b) one or more formulation excipients.

18. The formulation of paragraph 17, wherein the formulation adjuvant is a solid carrier, a liquid carrier, or a surface-active agent.
19. The formulation of paragraph 17 or paragraph 18, wherein the formulation is in the form of dusting powders, gels, wettable powders, water-dispersible granules, water-dispersible tablets, effervescent compressed tablets, emulsifiable concentrates, microemulsifiable concentrates, oil-in-water emulsions, oil flowables, aqueous dispersions, oil dispersions, suspoemulsions capsule suspensions, emulsifiable granules, soluble liquids, water-soluble concentrates (with water or a water miscible organic solvent as carrier), or impregnated polymer films.
20. A method for promoting plant growth comprising:

(i) applying one or more formulations to a plant, a plant part, or a growing site of plant,

wherein the one or more formulations comprise
(a) one or more compounds of any one of paragraphs 1-16, or salts thereof, and
(b) one or more excipients,

wherein the one or more compounds or salts thereof are in an effective amount to promote plant growth.

21. The method of paragraph 20, wherein the plant is a cereal, grain, or vegetable plant.
22. The method of paragraph 20 or paragraph 21, wherein the plant is wild-type or mutant.
23. The method of any one of paragraphs 20-22, wherein the plant part is seed or seedling.
24. The method of any one of paragraphs 20-22, wherein the growing site of plant is soil before, during, or after planting the plant.
25. The method of any one of paragraphs 20-22, wherein the growing site of plant is a growth medium.
26. The method of any one of paragraphs 20-25 further comprising a step of diluting the formulation in a liquid carrier prior to step (i).
27. The method of paragraph 26, wherein the liquid carrier is water.
28. The method of paragraph 26 or paragraph 27, wherein the concentration of the one or more compounds or salts thereof after dilution is between about 0.1 μM and about 1 mM.
29. The method of any one of paragraphs 20-28 further comprising applying one or more agriculturally beneficial agents prior to, during, or after step (i).
30. The method of paragraph 29, wherein the one or more agriculturally beneficial agents are fertilizers, micronutrients, microorganisms, or a combination thereof.
31. The method of any one of paragraphs 20-30, wherein the one or more compounds or salts thereof are in an effective amount to decrease biosynthesis and release of strigolactone plant hormones from the plant.
32. The method of paragraph 31, wherein the biosynthesis and release of strigolactone plant hormones from the plant is decreased by at least about 40% compared to an untreated plant under same conditions.
33. A method for inhibiting growth of a parasitic weed comprising:

(i) applying one or more formulations to the parasitic weed, a part of the parasitic weed, or a growing site of parasitic weed

wherein the one or more formulations comprise
(a) one or more compounds of any one of paragraphs 1-16, or salts thereof, and
(b) one or more excipients,

wherein the one or more compounds or salts thereof are in an effective amount to inhibit growth of the parasitic weed.

34. The method of paragraph 33, wherein the parasitic weed is a Striga species or an Orobanche species of the Orobanchaceae family.
35. The method of paragraph 33 or 34, wherein the growing site of the parasitic weed is soil before, during, or after planting a host plant.
36. The method of paragraph 35, wherein the growing site of the parasitic weed is soil after planting the host plant.
37. The method of any one of paragraphs 33-36 further comprising a step of diluting the formulation in a liquid carrier prior to step (i).
38. The method of paragraph 36, wherein the liquid carrier is water.
39. The method of paragraph 37, wherein the concentration of the one or more compounds or salt thereof after dilution is between about 0.1 μM and about 1 mM, preferably between about 1 μM and about 10 μM.
40. The method of any one of paragraphs 33-39 further comprising applying one or more herbicides prior to, during, or after step (i).
41. The method of any one of paragraphs 33-40, wherein the one or more compounds or salt thereof are in an effective amount to inhibit germination of a seed of the parasitic weed.

The present invention will be further understood by reference to the following non-limiting examples.

### EXAMPLES

The Examples below demonstrated highly effective compounds that promote plant growth. These exemplary compounds show high efficiency in accelerating plant growth, combating root parasitic weeds (e.g., Striga), and regulating plant architecture (i.e., number of branches, root type, and root growth), which, in some applications, exceeds that of natural plant metabolites, such as Zax. Further, these compounds are the first described synthetic compounds with Zaxinon (or “Zax”) activity.

### Example 1. Synthesis of Compounds MZ2-MZ5

Materials and Methods

General Procedure 1 (Wittig Reaction)

In a round-bottom flask, the mixture of dimethyl formamide (2.0 ml), aldehyde (1.0 mmol) and (acetylmethylene)triphenylphosphorane (4.0 mmol) was stirred for 2 h at 80° C., then ethyl acetate (15 ml) was added to the reaction mixture, which was washed with water and brine. The organic layer was dried over anhydrous sodium sulfate and concentrated under reduced pressure, then purified by column chromatography on silica gel (Wakosil®C-300HG), in which a mixture of hexane-ethylacetate was used as an eluent.

General Procedure 2 (Suzuki-Miyaura Cross Coupling)

In a round-bottom flask, the mixture of aryl bromide (2.0 mmol), boronic acid (1.0 mmol), THF (15.0 ml), 2N—Na₂CO₃ (4.5 ml) and tetrakis(triphenylphosphine)palladium(0) (0.01 mmol) were refluxed overnight with stirring, then THF was removed under reduced pressure. The resultant mixture was solved into ethylacetate (15 mml), which was washed with water and brine. The organic layer was dried over anhydrous sodium sulfate and concentrated under reduced pressure, then purified by column chromatography on silica gel (Wakosil®C-300HG), in which a mixture of hexane-ethyl acetate was used as an eluent.

General Procedure 3

In a round-bottom flask, 1M-boron tribromide in CH₂Cl₂ was added to the solution of methoxy aryl (1.0 mmol) in dichloromethane (5.0 ml) at 0° C. and stirred for 1 h, then quenched with water (10 ml), diluted with CH₂C₁₂ (10 ml), and washed with brine. The organic layer was dried over anhydrous sodium sulfate and concentrated under reduced pressure, then purified by column chromatography on silica gel (Wakosil®C-300HG), in which a mixture of hexane-ethylacetate was used as an eluent.

Chemical Data

MZ2 ((E)-4-(3-(4-hydroxyphenoxy)phenyl)but-3-en-2-one). 3-(4-Methoxyphenoxy)benzaldehyde was used as a starting material of procedure 3, then obtained compound was subjected to the procedure 1 to give MZ2 (44% yield).

1H NMR (500 MHz, CDCl3): δ 7.43 (1H, d, J=16.5 Hz), 7.32 (1H, t, J=8.0 Hz), 7.21 (1H, d, J=8.0 Hz), 7.07 (1H, s), 6.98 (1H, d, J=8.0 Hz), 6.94 (2H, d, J=8.5 Hz), 6.84 (2H, d, J=8.5 Hz), 6.63 (1H, d, J=16.5 Hz), 4.88 (1H, s), 2.36 (3H, s).

HRMS (ESI+) m/z=255.1016 calculated for C₁₆H1503 [M+H]+, found: 255.1011.

MZ3 ((E)-4-(3-(4-methoxyphenoxy)phenyl)but-3-en-2-one). 3-(4-Methoxyphenoxy)benzaldehyde was used as a starting material of procedure 1 to give MZ3 (81% yield).

1H NMR (500 MHz, CDCl3): δ 7.42 (1H, d, J=16.5 Hz), 7.30 (1H, t, J=8.0 Hz), 7.19 (1H, d, J=8.0 Hz), 7.06 (1H, s), 6.95-7.00 (3H, m), 6.89 (2H, d, J=7.0 Hz), 6.61 (1H, d, J=16.5 Hz), 6.63 (1H, d, J=16.5 Hz), 3.81 (1H, s), 2.34 (3H, s).

HRMS (ESI+) m/z=269.1172 calculated for C₁₇H1703 [M+H]+, found: 269.1172.

MZ4 (1-(4″-hydroxy-[1,1′:3′,1″-terphenyl]-3-yl)ethan-1-one). 1,3-Dibromobenzene was used as a starting material of procedure2, in which 4-hydroxyphenylboronic acid and 3-acetylphenylboronic acid were sequentially subjected to the cross coupling to give MZ5, which was then subjected to the procedure 4 to give MZ4 (11% yield).

1H NMR (500 MHz, CDCl3): δ 8.23 (1H, s), 7.96 (1H, d, J=8.0 Hz), 7.40 (1H, d, J=8.0 Hz), 7.76 (1H, s), 7.49-7.59 (6H, m), 6.94 (2H, d, J=9.0 Hz), 2.68 (3H, s).

HRMS (ESI+) m/z=289.1223 calculated for C₂₀H1702 [M+H]+, found: 289.1232.

MZ5 (1-(4″-methoxy-[1,1′:3,1″-terphenyl]-3-yl)ethan-1-one). 1,3-Dibromobenzene was used as a starting material of procedure 2, in which 4-hydroxyphenylboronic acid and 3-acetylphenylboronic acid were sequentially subjected to the cross coupling to give MZ5 (75% yield).

1H NMR (500 MHz, CDCl3): δ 8.23 (1H, s), 7.96 (1H, d, J=8.0 Hz), 7.85 (1H, d, J=7.5 Hz), 7.78 (1H, s), 7.50-7.61 (6H, m), 7.01 (2H, d, J=6.5 Hz), 3.87 (3H, s), 2.67 (3H, s).

HRMS (ESI+) m/z=303.1380 calculated for C₂₁H1902 [M+H]+, found: 303.1376.

Results

### Scheme of the Synthesis:

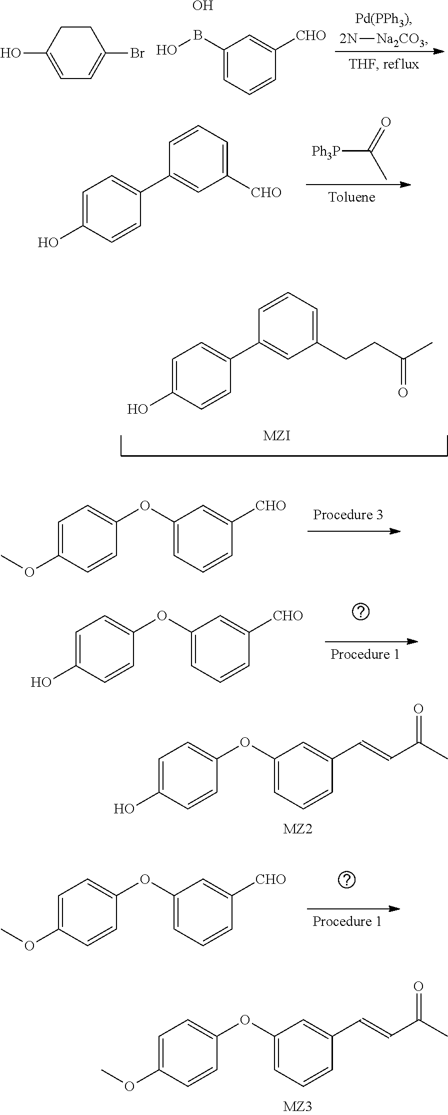

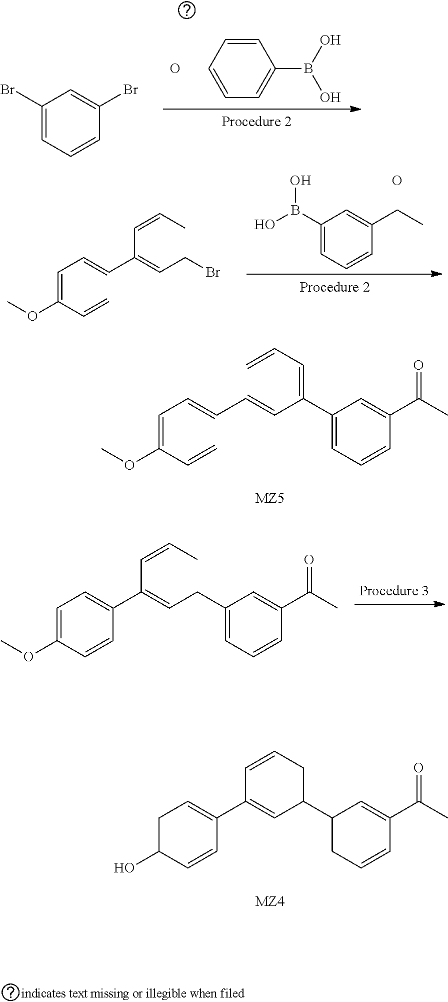

The synthesis of zaxinone takes 4-5 steps with relative low yield.

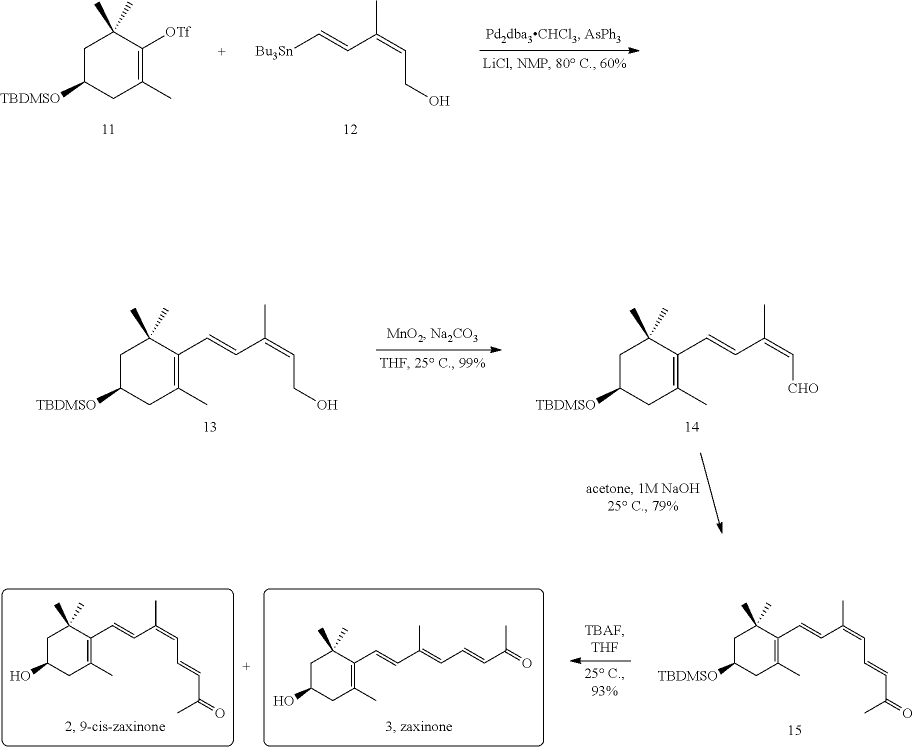

In contrast, the synthetic path of MZ is simple (only one or two steps), which hugely lowers the cost and chemical waste. The synthesis produces the compounds with high yield.

### Example 2. Screening of Compounds with Zax Activity

Materials and Methods

Rice Plant Material and Growth Conditions

Rice plants were grown under controlled conditions (a 12 h photoperiod, 200-μmol photons m⁻² s⁻¹ and day/night temperature of 27/25° C.). Rice seeds were surface-sterilized in 50% sodium hypochlorite solution with 0.01% Tween-20 for 15 min. The seeds were rinsed with sterile water and germinated in the dark. The pre-germinated seeds were transferred to petri dishes containing half-strength liquid Murashige and Skoog (MS) medium and incubated in a biochamber for 7 days. Thereafter, the seedlings were transferred into black falcon tubes filled with half-strength modified Hoagland nutrient solution with adjusted pH to 5.8. The nutrient solution consisted of 5.6 mM NH₄NO₃, 0.8 mM MgSO₄O.7H₂O, 0.8 mM K₂SO₄, 0.18 mM FeSO₄.7H₂O, 0.18 mM Na₂EDTA.2H₂O, 1.6 mM CaCl₂.2H₂O, 0.8 mM KNO₃, 0.023 mM H₃BO₃, 0.0045 mM MnCl₂.4H₂O, 0.0003 mM CuSO₄.5H₂O, 0.0015 mM ZnCl₂, 0.0001 mM Na₂MoO₄.2H₂O and with or without 0.4 mM K₂HPO₄.2H₂O, resulting in the +Pi and −Pi conditions, respectively.

Transcript Analysis

For transcript analysis, one-week-old seedlings were grown hydroponically in half-strength modified Hoagland nutrient solution with K₂HPO4.2H₂O (+Pi) and without K₂HPO₄.2H₂O (+Pi) (−Pi) for 7 days. Seedlings were further treated with zaxinone or MZ compounds for 6 h, and tissues were collected.

SL Analysis

For SL analysis, one-week-old seedlings were transferred into 50 ml falcon tubes (two seedlings per tube), containing half-strength Hoagland nutrient solution with K₂HPO4.2H₂O (+Pi), and grown in the biochamber for one week. Rice seedlings were then subjected to phosphate deficiency (−Pi) for another one week. On the day of root exudates collection, rice seedlings were first treated with 5 μM Zaxinone, or MZ compounds for 6 h, and then root exudates and root tissues were collected from each tube for LC-MS/MS analysis and Striga bioassays.

Gene Expression Analysis

Roots of rice seedling were ground and homogenized in liquid nitrogen, and total RNA was isolated using a Direct-zol RNA Miniprep Plus Kit following the manufacturer's instructions (ZYMO RESEARCH; USA). cDNA was synthesized from 1 μg of total RNA using iScript cDNA Synthesis Kit (BIO-RAD Laboratories, Inc, 2000 Alfred Nobel Drive, Hercules, Calif.; USA) according to the instructions in the user manual. The gene expression level was detected by real-time quantitative RT-PCR (qRT-PCR) which was performed using SYBR Green Master Mix (Applied Biosystems; www.lifetechnologies.com) in a CFX384 Touch™ Real-Time PCR Detection System (BIO-RAD Laboratories, Inc, 2000 Alfred Nobel Drive, Hercules, Calif.; USA). Primers used for qRT-PCR analysis are listed in Table 1. The gene expression level was calculated by normalization of a housekeeping gene in rice, Ubiquitin (OsUBQ) (Table 1). The relative gene expression level was calculated according to 2-^{ΔΔ}CT method.

TABLE 1
Primer sequences used in this study

| Experiment | Primer name | Sequence (5′-3′) |
|---|---|---|
| qRT-PCR | OsUbi-Q-F | GCCCAAGAAGAAGATCAAGAAC |
| | | (SEQ ID NO: 1) |
| | OsUbi-Q-R | AGATAACAACGGAAGCATAAAAG |
| | | (SEQ ID NO: 2) |
| | OsD27-RT2-F | CTTCCAAGCTACATCCTCAC |
| | | (SEQ ID NO: 3) |
| | OsD27-RT2-R | CCCAACCAACCAAGGAAA |
| | | (SEQ ID NO: 4) |
| | OsCCD7-RT1-F | CAGTCTCCAAGCACAGATG |
| | | (SEQ ID NO: 5) |
| | OsCCD7-RT1-R | GTTCTTTGGCACCTCTAGTT |
| | | (SEQ ID NO: 6) |
| | OsCCD8b- | TGGCGATATCGATGGTGA |
| | RT2-F | (SEQ ID NO: 7) |
| | OsCCD8b- | GACCTCCTCGAACGTCTT |
| | RT2-R | (SEQ ID NO: 8) |
| | OsMax1-900 | ATTGTCAGCGATCCACTTC |
| | qPCR F2 | (SEQ ID NO: 9) |
| | OsMax1-900 | GCCCCGTTCTTGAAATTG |
| | qPCR R2 | (SEQ ID NO: 10) |
| | OsRubQ1 F | GGGTTCACAAGTCTGCCTATTTG |
| | | (SEQ ID NO: 11) |
| | OsRubQ1 R | ACGGGACACGACCAAGGA |
| | | (SEQ ID NO: 12) |
| | OSPt11 F | GAGAAGTTCCCTGCTTCAAGCA |
| | | (SEQ ID NO: 13) |
| | OSPt11 R | CATATCCCAGATGAGCGTATCATG |
| | | (SEQ ID NO: 14) |
| | OsLysM F | CGCTGACATGCAACAAGGTG |
| | | (SEQ ID NO: 15) |
| | OsLysM R | CTTCGCGCAGTTGATGTTTGG |
| | | (SEQ ID NO: 16) |
| | 18SF.m F | CCTTTTGAGCTCGGTCTCGTG |
| | | (SEQ ID NO: 17) |
| | 18SF.m R | TGGTCCGTGTTTCAAGACG |
| | | (SEQ ID NO: 18) |

Chemical Stability

MZ3, and MZ5 were tested for their chemical stability at 21±1° C. with pH 5.0-6.0 aqueous solution following the protocol described recently. 50 μl of each compound solutions (1 mg ml-1) was prepared with 175 μl ethanol and 750 μl Mili-Q water. Thereafter, 25 μl Indanol (1 mg ml-1, internal standard) was spiked in 975 μl previous prepared solution. The degradation was monitored at a time course by UPLC analysis using an Agilent HPLC ZORBAX Eclipse XDB-C18 column (3.5 μm, 4.6×150 mm), eluted first by 5% acetonitrile in water for 0.5 min then by a gradient flow from 5% to 100% acetonitrile within 18 min, and by 100% acetonitrile for 5 min. The column was operated at 40° C. at 0.35 ml min⁻¹ flow rate. Compounds eluted from the column were detected with a photodiode array detector, and the relative quantity of non-degraded amount was calculated by integration comparison with Indanol.

Zaxinone was tested under the same conditions for comparison. Synthetic zaxinone was purchased (customized synthesis) from Buchem B.V. (Apeldoorn, The Netherlands).

Striga hermonthica Seed Germination Bioassays

Striga hermonthica seed germination bioassays were conducted by following the procedure as described previously. About 50-100 sterilized Striga seeds were spread on a glass fiber filter paper disc. Then 12 discs with Striga seeds were put on a sterilized filter paper, moistened with 3 ml sterilized water in a 90 mm petri plate. The plates were sealed with parafilm and cover with aluminum foil. All the plates were kept at 30° C. for 10 days for pre-conditioning. On 11^{th} day, six dry discs with pre-conditioned seeds were selected and put in a 90 mm petri-dish. A filter paper ring moistened with 0.9 ml sterile MilliQ water was placed in the plate. The root exudates collected from Zaxinone or MZ compounds treated rice seedlings was applied at 50 μl on each six disc. Sterile MilliQ water and standard SL analog GR24 (2.5 μM) were included as a negative and positive control, respectively. After application, Striga seeds were incubated in dark at 30° C. for 24 hours. Germination (seeds with radicle emerging through seed coat) was scored under a binocular microscope, and germination rate (%) was calculated.

Statistical Analysis

All of the experiments were performed with at least three biological replicates each. Statistical tests were carried out through One-way analysis of variance (One-way ANOVA) and Tukey's post hoc test or via two-tailed Student's t tests or by non-parametric Kruskal-Wallis test, using a probability level of P<0.05. All statistical elaborations were performed using R program or GraphPad Prism Version 8.

Data Availability

All data generated or analyzed during this study are included in this published article and its supplementary information files.

Results

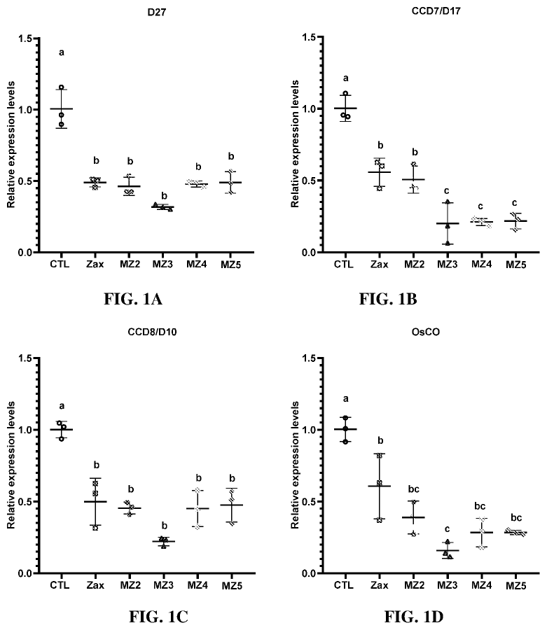

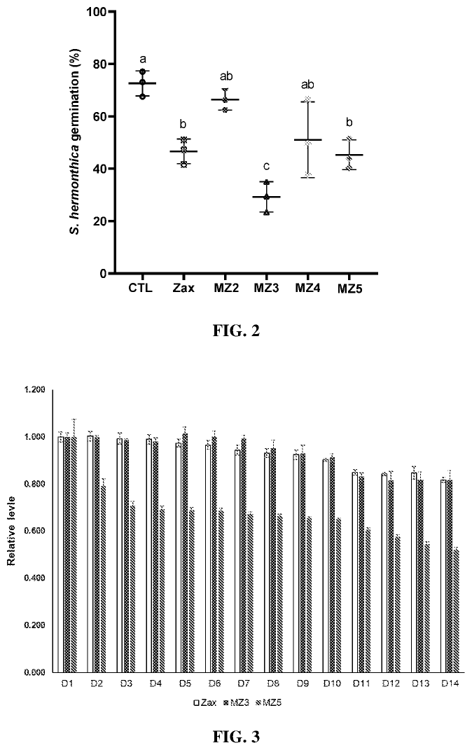

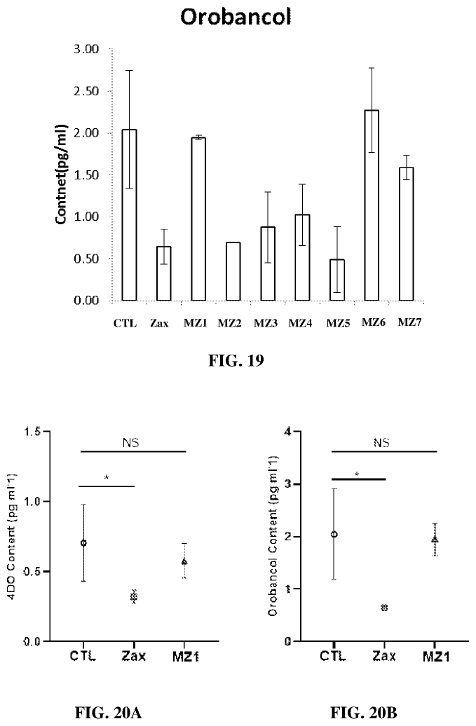

To screen the effect of MZ compounds on SL biosynthesis, the Striga seed germination activity of collected exudates from the same samples was determined. This germination assay is used as a biological tool for the perception of total released SLs. The transcript levels of the SL biosynthesis enzymes DWARF27 (D27), CCD7 (D17), CCD8 (D10) and the 4-deoxyorobanchol-forming carlactone oxidase (CO) in wild-type seedlings exposed to Pi-starvation and upon treatment with zaxinone and MZ1-MZ7 (5 μM) were measured. The compounds MZ1, MZ6 and MZ7 did not show significant activity in the SL level of the root exudate when compared with Zaxinone, while MZ3 and MZ5 exerted an obvious effect on zaxinone targets (FIGS. 19 and 20A-20B). MZ1 contains 7 carbon-side-chain. The results show that the side chain length should be optimal C₈. MZ compounds (MZ2-MZ5) with C₈ side chain length were synthesized following the procedure depicted in the scheme in Example 1. The yield is 44%; 81%; 11%; and 75% for MZ2, MZ3, MZ4, and MZ5 respectively. Although all MZ compounds treatment decreased the transcripts level, MZ3 showed the strongest inhibition compared to the others (FIGS. 1A-1D). Intriguingly, the MZ2 and MZ4 did not suppress Striga seed germination while they were still downregulating the SL biosynthetic genes. On the contrary, Zaxinone, MZ3, and MZ5 significantly decreased the germination rate around 29%, 45%, and 46% respectively (FIG. 2), which in line with the observation that MZ3 strongly lowered SL biosynthesis and release. Considering the structure difference of MZ2 and MZ3, the methoxy group at 3′-hydroxyl group (—OH) increases the negative regulation on SL genes expression, which suggests natural Zaxinone may be further metabolized at this position in plants. In addition, the structure between MZ3 (ether) and MZ5 (benzene) are different at the chain-modification, and it can be that the oxygen makes MZ3 more active and stable.

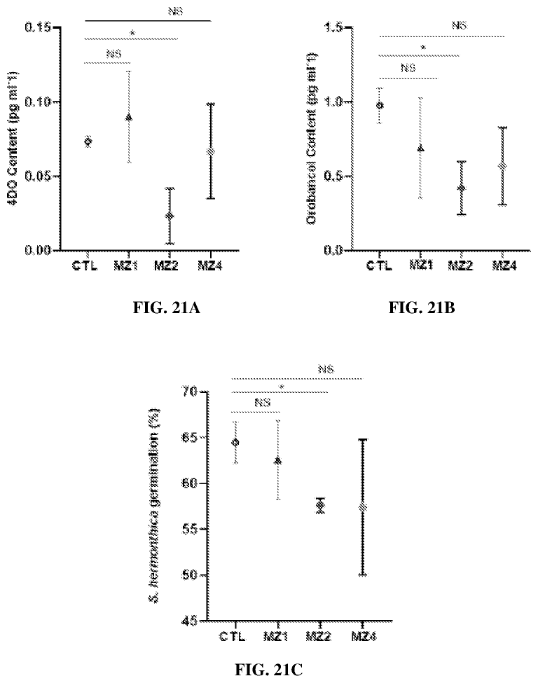

In comparison with MZ1 and mock control, application of MZ2 led to a significant decrease in SL level and Striga germination rate, while MZ4 showed a tendency to reduce SLs, particularly orobanchol, release (FIGS. 21A-21C). Besides a common chain length, MZ3 and MZ5 contain a methoxy group instead of the hydroxy group at C₃ in zaxinone and the corresponding position in MZ2 and MZ4. Comparison of the effect of MZ2 and MZ3, and of MZ4 and MZ5, on Striga seed-germinating activity demonstrated that this methylation has a positive effect on the activity of zaxinone mimics (FIG. 2). Zaxinone may be converted into methyl-zaxinone in planta. Methyl-zaxinone was synthesized and its presence was tested in planta as well as its biological activity. However, no methyl-zaxinone was detected in rice plants (data not shown). In addition, the biological efficiency of methyl-zaxinone in inducing Striga seed germination was similar to that of Zaxinone (data not shown), showing that the presence of the methoxy group per se is not the reason of the increased activity of MZ3 and MZ5 and that direct zaxinone methylation might not take place in planta. Supporting the latter conclusion, a conversion of MZ2 or MZ4 into MZ3 or MZ5, respectively, was not detected in rice plants fed with the former two mimics using LC-MS analysis (data not shown). These data shows that the higher activity observed with MZ3 and MZ5 could be a result of increased hydrophobicity caused by the methyl group, which may improve their uptake and transport. MZ3 and MZ5 in shoots of rice plants fed with these compounds through roots were detected using LC-MS analysis (data not shown). A positive effect of the presence of a phenoxy group in MZ3 instead of the unmodified phenyl ring in MZ5 was also observed. This difference might be due to an increased stability caused by a shorter conjugated double bond system and/or the ether bond.

To determine the stability of MZ3 and MZ5 compared to zaxinone, a 14-day HPLC analysis under room temperature was performed. MZ3 is much more stable than MZ5 (FIG. 3), confirming the oxygen-dependent stability and activity. Moreover, MZ2 and MZ4 reduced the SL biosynthetic genes expression, but they did not affect the SL release in the exudate, which can be associated to with complex transportation systems of SLs.

### Example 3. The Compounds Positively Regulate Plant Growth and Development

Materials and Methods

Rescuing Zas Mutant with MZ3 and MZ5

For rescuing zas mutant phenotypes and investigating the effect of the compounds MZ3 and MZ5 on different genotypes, one week-old seedlings were grown hydroponically in half-strength modified Hoagland nutrient solution containing K₂HPO₄.2H₂O (+Pi), 2.5 μM and 5 μM zaxinone or compounds (MZ2-MZ5) (dissolved in 0.1% acetone) or the corresponding volume of the solvent (control) for 21 days (i.e. 3 weeks). The solution was changed every other day, adding the chemical at each renewal.

Testing Effect of MZ3 and MZ5 on Rice Phenotypes

For testing MZ3 and MZ5 on rice phenotypes in the rhizotron system (48 cm×24 cm×5 cm), three-day-old seedlings were grown in soil with half-strength modified Hoagland nutrient solution containing 0.4 mM K₂HPO₄.2H₂O (+Pi) and 5 μM zaxinone for two weeks. The solution was changed every other day, adding the chemical at each renewal. To increase the compounds stability, 1 μl/ml emulsifier (cyclohexanone+Atlas G1086 kindly provided by Mr. Han Rieffe of CRODA, Gouda; The Netherlands) was added into Hoagland nutrient solution. Root surface area was analyzed with the ImageJ software.

Testing Effect of MZ3 and MZ5 on Tomato Plant

Tomato seeds (c.v. Money maker) were sterilized and pre-germinated on moistened filter paper at 22° C. for 4-5 days, transferred to light for 3-4 days or direct sowed of seeds from fresh tomatoes in soils at 25° C. Most uniform seedlings (10 days old) were selected and transferred to pots. After one week of the transfer, 5 μM compounds were used for foliar application. Each compound was applied three times in a week up to four weeks: 20 mL per pot (spray as runoff) in first 3 weeks, and 40 mL per pot in last 4th week. The plants were grown under normal growth condition (27° C., 70% RH).

Morphometric Analysis

Non mycorrhizal and mycorrhizal WT, WT plants treated with 5 μM or 50 nM MZ3 or MZ5 were collected 35 days post inoculation (dpg). For each plant shoot and root fresh weights, shoot length, leaves numbers, crown roots (CR) number and length.

Results

To understand the biological roles of MZ compounds and their activities on plant growth and development, the most active ones, MZ3 and MZ5, were selected for further testing. Both MZ3 and MZ5 present the methoxy group adjacent to the ring.

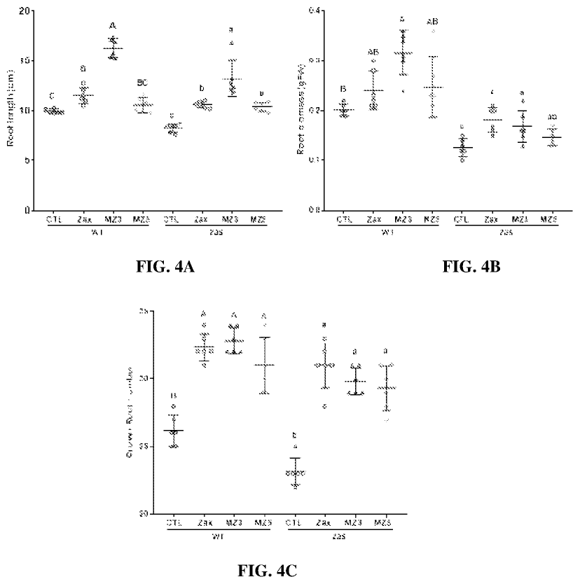

To determine whether these compounds are functionally comparable to zaxinone, they had to rescue zas mutant phenotype and to promote wild-type rice plant growth. zas mutant and the corresponding Nipponbare wild-type seedlings were supplied with 2.5 μM exogenous MZ compounds in the hydroponic system for up to three weeks, respectively. This treatment, indeed, increased the root length, number of crown roots, and roots biomass phenotypes in zas mutant together with boosting WT plant growth (e.g. triggered root growth of wild-type plants), which demonstrated the same biological effects as zaxinone. Moreover, MZ3 was more active than zaxinone in increasing the root length of wild-type plants in the hydroponic system (FIGS. 4A-4C).

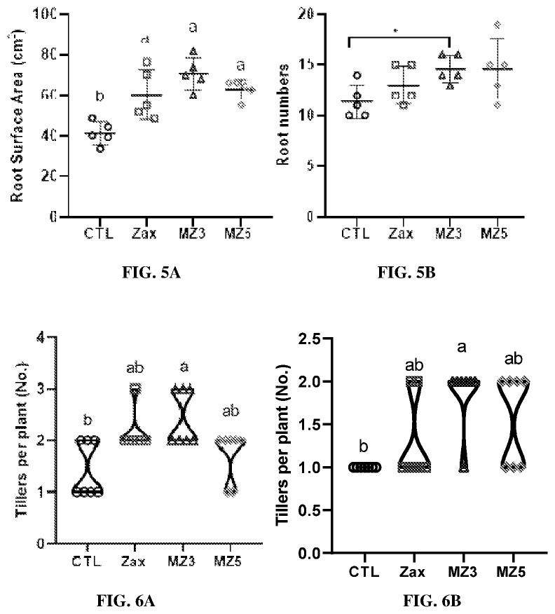

The effect of the two MZ compounds in soil was tested at a 5 mM concentration, using the rhizotron system and in comparison with Zaxinone. In soil, the application of 5 μM MZ3 and MZ5 increased more the root surface area and root numbers of WT plants compared with zaxinone treatment, respectively (FIGS. 5A and 5B). Tillering is a SL-dependent developmental process affected by Zaxinone, as shown for the zas mutant. A 14-day treatment of the MZ compounds increased tillering numbers in Nipponbare as well as in high SL producer IAC-165 rice cultivar. These results show that the regulation of MZ compounds on plant architecture is associated with SL pathway (FIGS. 6A and 6B).

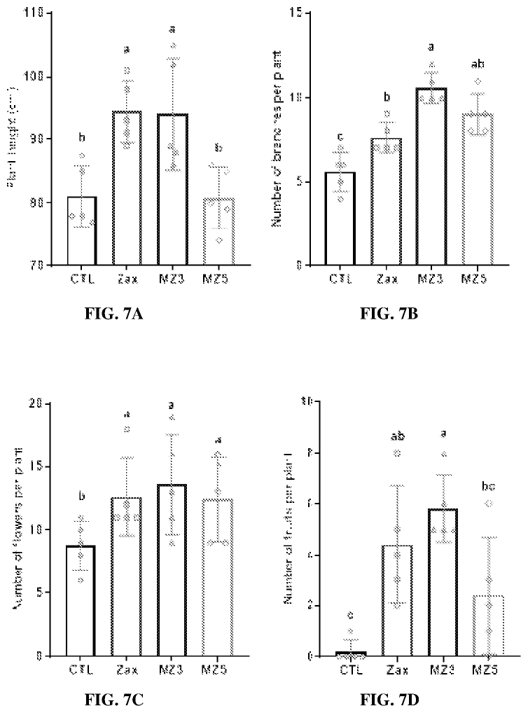

Furthermore, in order to widen the use of these compounds, the effect of 5 μM MZ compounds on tomato plant was investigated by foliar application. Results show that MZ3 strongly increased the plant height, branch, flowers and fruit numbers compared to zaxinone (FIGS. 7A-7D).

These data demonstrates that MZ3 and MZ5 are positive regulators of plant growth, enhancing plant performance at low concentrations, with more robust effects than zaxinone, which provides the insight for the agriculture applications.

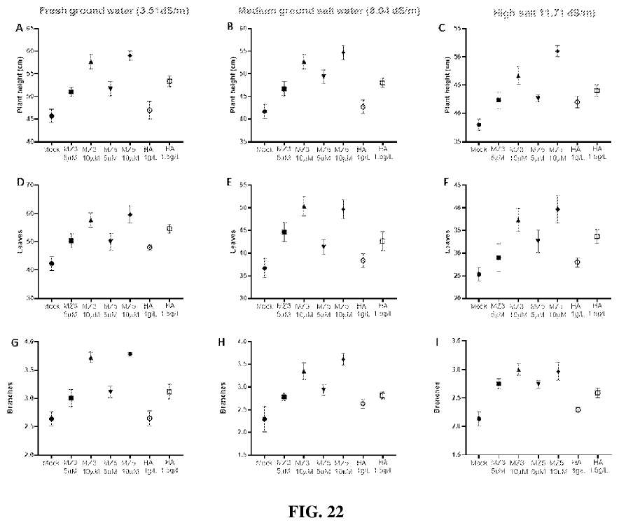

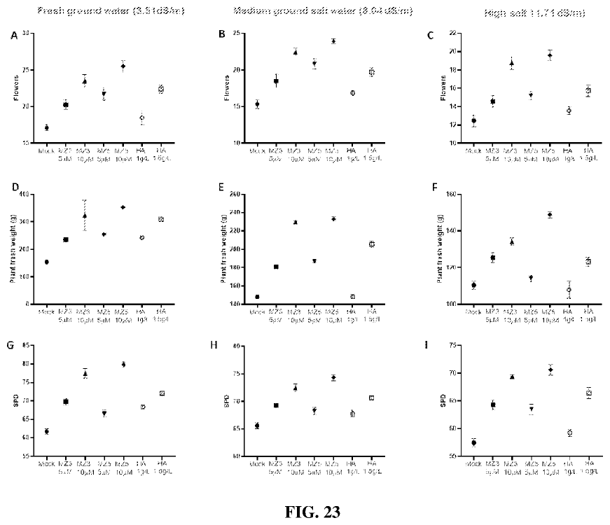

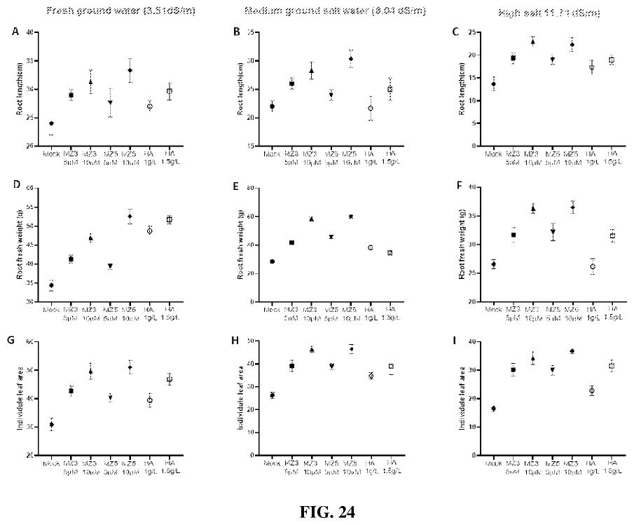

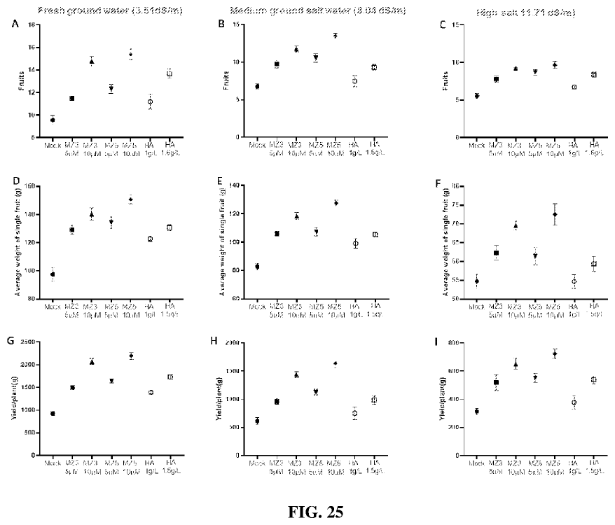

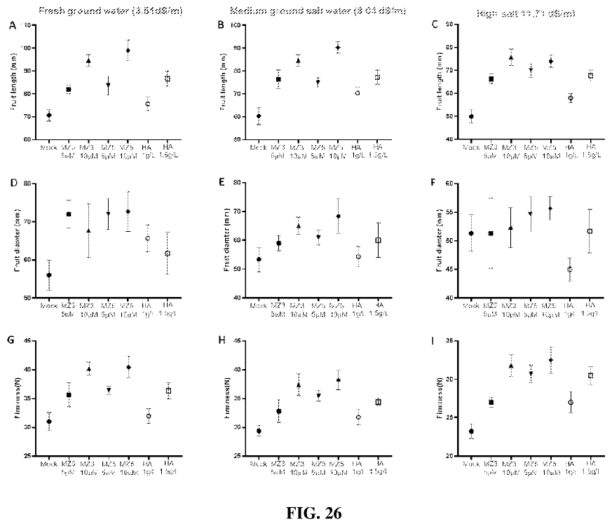

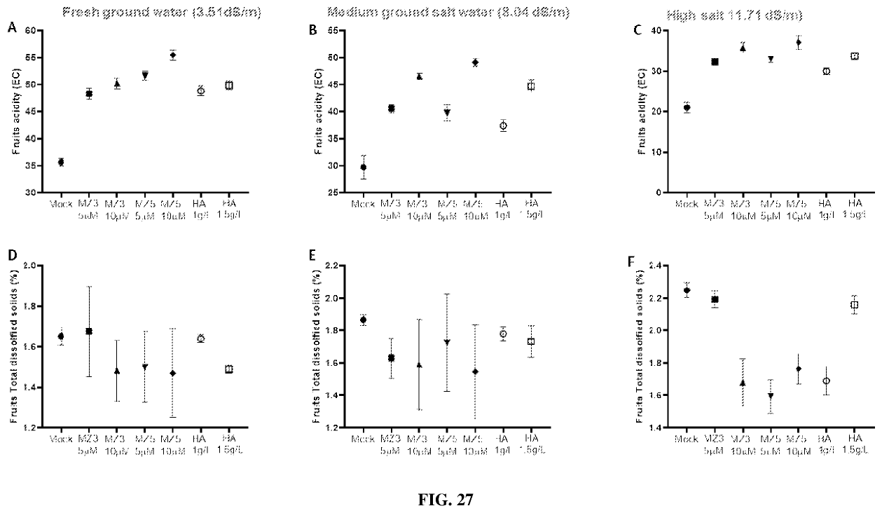

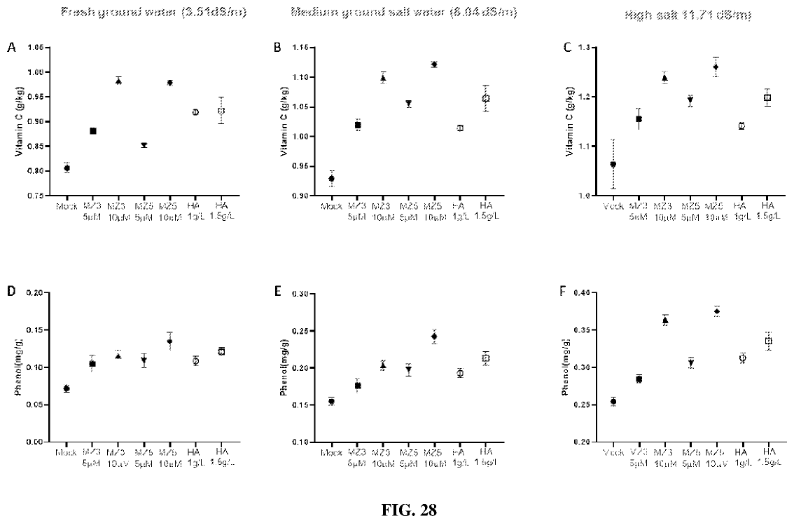

A field study on green pepper was conducted and the results are shown in FIGS. 22A-22I, 23A-23I, 24A-24I, 25A-25I, 26A-26I, 27A-27F, and 28A-28F. The field study data demonstrates significant increase of the plant height, leave number and individual leave area, branch number, number of flower, plant fresh weight, root length, root fresh weight, number of fruit produced, quality of produced fruit (e.g. fruit weight, fruit size, firmness, acidity, vitamin C and phenol contents), and the yield.

### Example 4. The Compounds Reduce Strigolactones (SLs) Biosynthesis and Release

Materials and Methods

Quantification of SLs in Rice Root Exudates

For the quantification of SLs in rice root exudates, 50 ml of root exudates spiked with 0.672 ng of D₆-5-deoxystrigol, was brought on a 500 mg/3 ml fast SPE C18 column preconditioned with 6 ml of methanol and 3 ml of water. After washing with 3 ml of water, SLs were eluted with 5 ml of acetone. The 4-deoxyorobanchol fraction (acetone-water solution) was concentrated to SL aqueous solution (˜500 μl), followed by the extraction with 1 ml of ethyl acetate. 750 μl of SLs enriched organic phase was then transferred to 1.5 ml tube and evaporated to dryness under vacuum. The dried extract was dissolved in 100 μl of acetonitrile:water (25:75, v:v) and filtered through a 0.22 m filter for LC-MS/MS analysis.

Quantification of SLs in Rice Root Tissues

For the quantification of SLs in rice root, plant tissue material was lyophilized and grinded. Approximately 20 mg root tissue spiked with 0.672 ng of D₆-5-deoxystrigol was extracted with 2 ml of ethyl acetate in an ultrasound bath (Branson 3510 ultrasonic bath) for 15 min, followed by centrifugation for 8 min at 3800 rpm at 4° C. The supernatant was collected and the pellet was re-extracted with 2 ml of ethyl acetate. Then the two supernatants were combined and dried under vacuum. The residue was dissolved in 100 μl of ethyl acetate and 2 ml of hexane. The resulting extract solution was loaded on a Silica gel SPE column (500 mg/3 ml) preconditioned with 3 ml of ethyl acetate and 3 ml of hexane. After washing with 3 ml of hexane, SLs were eluted in 3 ml of ethyl acetate and evaporated to dryness under vacuum. The residue was re-dissolved in 200 μl of acetonitrile:water (25:75, v:v) and filtered through a 0.22 m filter for LC-MS/MS analysis. 4-deoxyorobanchol was analyzed by using HPLC-Q-Trap-MS/MS with MRM mode. Chromatographic separation was achieved on an Agilent 1200 HPLC system with a ZORBAX Eclipse plus C18 column (150×2.1 mm; 3.5 m; Agilent). Mobile phases consisted of water: acetonitrile (95:5, v:v, A) and acetonitrile (B), both containing 0.1% formic acid. A linear gradient was optimized as follows (flow rate, 0.2 ml/min): 0-10 min, 25% to 100% B, followed by washing with 100% B and equilibration with 25% B. The injection volume was 5 μl and the column temperature was maintained at 30° C. for each run. The MS parameters were listed as follows: positive ion mode, ion source of turbo spray, ion spray voltage of 5500 V, curtain gas of 20 psi, collision gas of medium, gas 1 of 80 psi, gas 2 of 70 psi, turbo gas temperature of 400° C., declustering potential of 60 V, entrance potential of 10 V, collision energy of 20 eV, collision cell exit potential of 15 V. The characteristic MRM transitions (precursor ion→product ion) were 331→216, 331→97 for 4-deoxyorobanchol; 347.1→233, 347.1→97 for orobanchol; 337→222, 337→97 for D₆-5-deoxystrigol.

Results

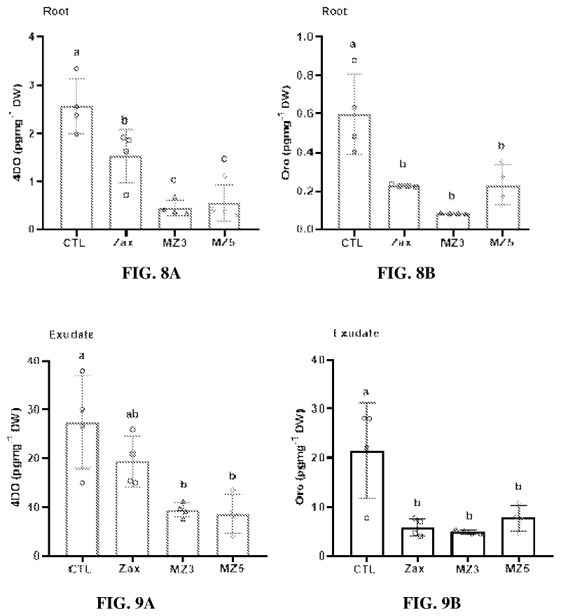

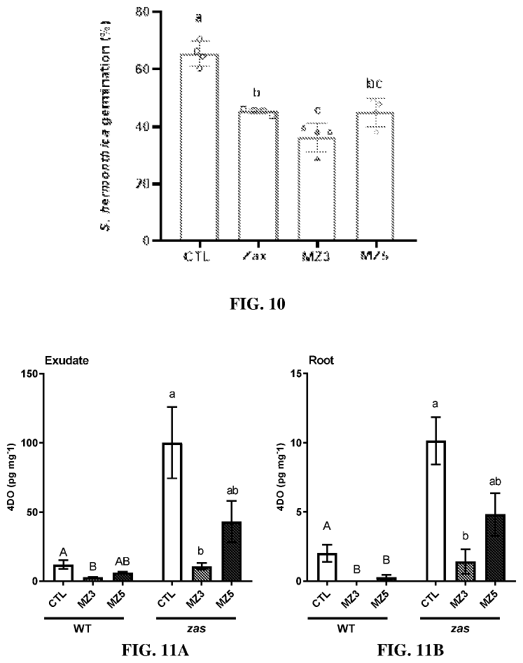

The increased tillering and branches numbers upon MZ application demonstrated an alteration of the SL biosynthesis and release in the investigated plants. The SLs (4-deoxyorobanchol and orobanchol) content in root tissues and exudates of hydroponically grown Pi-starved WT seedlings were exposed to 5 μM MZ3 and MZ5 for 6 hours, and then measured using LC-MS/MS. The Striga seed germination activity of the collected exudates was also tested. Although MZ5 reduced SL levels more than zaxinone, MZ3 treatment significantly decreased SL content in both roots (4DO, from 2.5 to 0.5 pgmg-1dry-root-weight; Oro, from 0.6 to 0.1 pgmg-1dry-root-weight) and root exudates (4DO, from 25 to 10 pgmg-1dry-root-weight; Oro, from 20 to 5 pgmg-1dry-root-weight), and lowered the germinating activity by around 40% (FIGS. 8A-8B, 9A-9B, and 10). In addition, MZ3 also strongly inhibited SL content in zas mutant compared to MZ5 (FIGS. 11A-11B), which was in agreement with the strong rescuing effects of zas phenotype upon MZ3 treatment (FIGS. 4A-4C). The effect of MZ3 or MZ5 was similar to that of Zaxinone and even significantly stronger in the case of 4-deoxyorobanchol. The two mimics, particularly MZ3, rescued the high SL phenotype of the rice zas mutant.

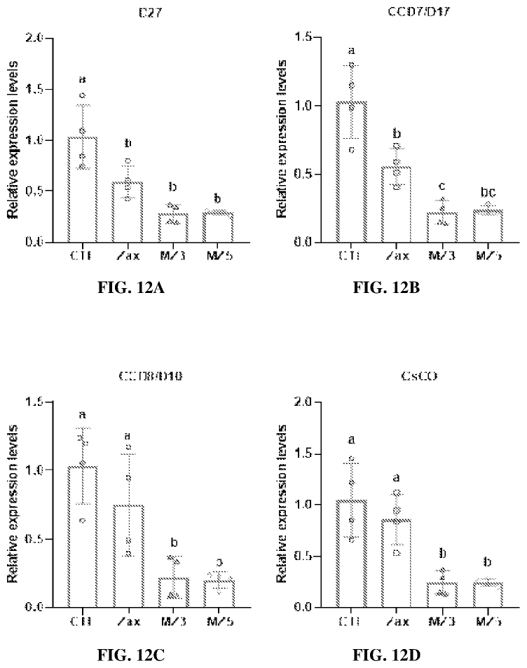

Next, the transcript levels of the SL biosynthesis enzymes D27, CCD7, CCD8 and the 4-deoxyorobanchol-forming carlactone oxidase (CO) from the same experiment were determined. Both MZ3 and MZ5 showed substantial genes suppressed effects (FIGS. 12A-12D). Application of MZ3 and MZ5 led to a pronounced decrease in the transcript level of the four enzymes, which was—at least in the case of CCD7 and CO transcripts-significantly lower than that observed with Zaxinone.

### Example 5. The Compounds Combat Striga

Materials and Methods

Striga hermonthica Infection in Rice

Pots experiment was conducted and slightly adapted from the protocol as described previously About 20 mg (ca 8000) Striga seeds were thoroughly mixed in 1.5 L sand and soil mixture (1:1) and added in 3 L perforated plastic pot containing 0.5 L clean soil in the bottom. The pots were kept in greenhouse-controlled conditions at 35° C. under moisture to preconditioned Striga seeds for 10 days. On 11th day, one week old five rice seedlings (cv IAC165) were planted in each pot. After three days of rice planting, each pot was first irrigated with 250 ml phosphorus deficient Hoaglands nutrient solution and after four hours of irrigation 25 ml formulated MZ3, MZ5 and Zaxinone (100 μM) were sprayed. Next day, each pot was again supplied with 250 ml nutrient solution to make the final concentration of each compound to 5 μM and to move the compound to rice seedlings. Pots with and without Striga seeds were kept as the negative and positive control. Each compound was applied twice in a week up to four weeks. The rice plants were grown under normal growth condition (30° C., 70% RH) for eight weeks. The number of emerged Striga plants in each pot were counted.

Results

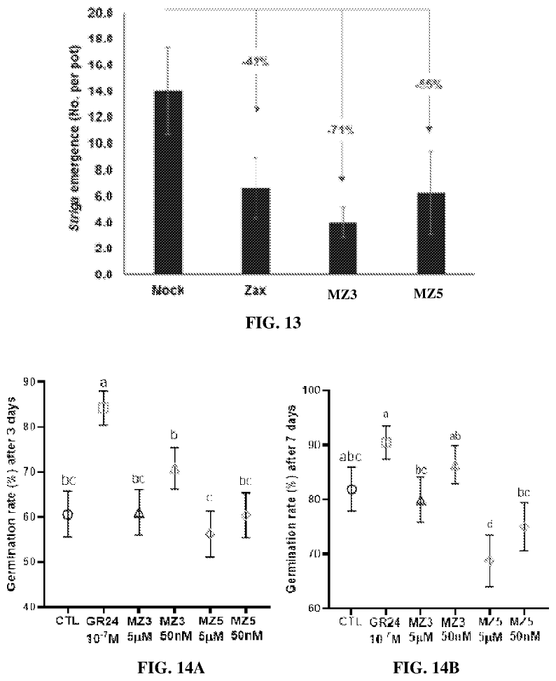

Apart from the potential application of MZ compounds to increase crop growth, this compound might be also utilized to combat root parasitic weeds as MZ compounds reduced SL biosynthesis and release. MZ3 and MZ5 were applied at 5 μM concentration to the Striga susceptible rice cv, respectively. IAC-165 plants grown in pots with Striga infested soil to make the same growth conditions in field. MZ3 and MZ5 clearly reduced the number of emerging Striga plants compared to the untreated control, with the highest reduction observed with MiZax3 (71%), followed by MiZax5 (55%) and zaxinone (42%) (FIG. 13). The reducing Striga emergence rate of MZ compounds provides a new insight for dealing with agriculture problems; however, chemical application in agriculture should also consider environment-friendly for the ecosystem, especially the plant-arbuscular mycorrhizal (AM) symbiosis.

### Example 6. The Compounds does not Affect Spores Germination

Materials and Methods

Plant and Fungal Material and Treatments

Seeds of WT plants and Nipponbare were germinated in pots containing sand and incubated for 10 days in a growth chamber under a 14 h light (23° C.)/10 h dark (21° C.). Plants used for mycorrhization were inoculated with Funneliformis mosseae (BEG 12, MycAgroLab, France). Fungus inoculum (25%) were mixed with sterile quartz sand and used for colonization. Plants were watered with a modified Long-Ashton (LA) solution containing 3.2 μM Na₂HPO₄.12H₂O (Hewitt, 1966) and were grown in a growth chamber under 14 h light (24° C.)/10 h dark (20° C.) regime. A set of WT plants were treated with 5 μM or 50 nM MZ3 or MZ5, by applying molecules twice a week directly in the nutrient solution.

Wild-type and WT treated mycorrhizal roots were stained with 0.1% cotton blue in lactic acid and the estimation of mycorrhizal parameters was performed by Trouvelot method and using MYCOCALC (http://www2.dijon.inra.fr/mychintec/Mycocalc-prg/download.html). Four parameters were considered: F %, percentage of segments showing internal colonization (frequency of mycorrhization); M %, average percentage of colonization of root segments (intensity of mycorrhization); a %, percentage of arbuscules within infected areas; A %, percentage of arbuscules in the whole root system.

For the molecular analyses, roots were immediately frozen in liquid nitrogen and stored at −80° C.

Gigaspora margarita Spores Germination

Spores were sterilized in a solution of Streptomycine sulphate (0.03% W/V) and Chloramine T (3% W/V) and germinated in 200 μl of MZ3 and MZ5 at 5 μM or 50 nM, GR24 10 nM or a solution of Water/Acetone (12.5 μl acetone in 25 ml water). For each treatments 96 sterilized spores were placed individually in the wells of a multi-well plate and treated with freshly prepared solutions at the beginning of the experiment and after 3 days. Spores were germinated in the dark at 30° C. and the germination rate was evaluated after 3 days and 7 days.

Results

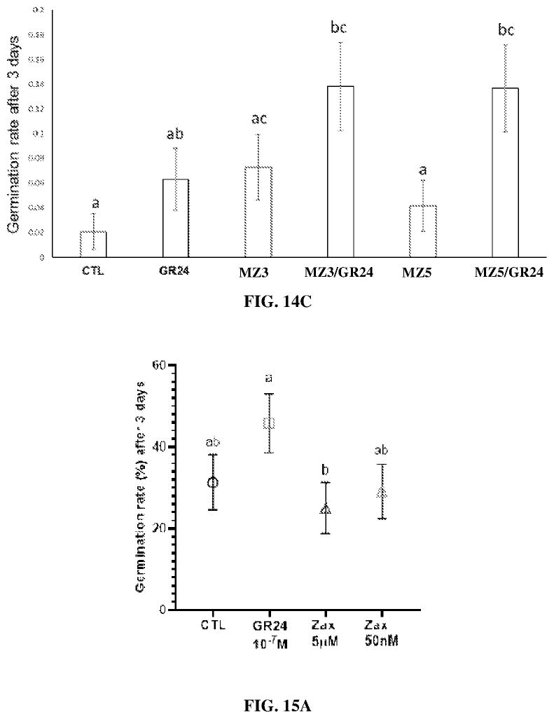

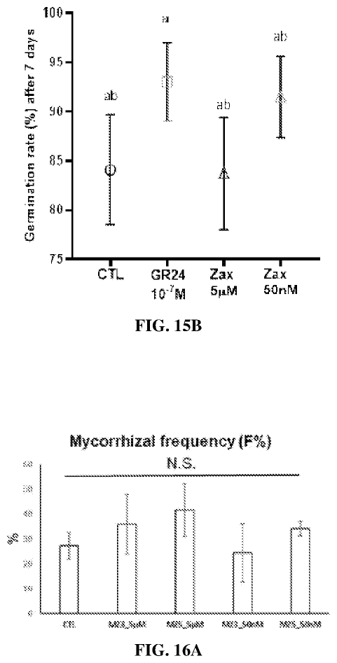

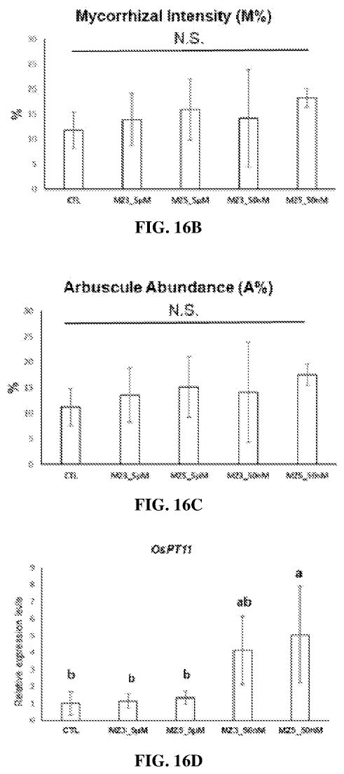

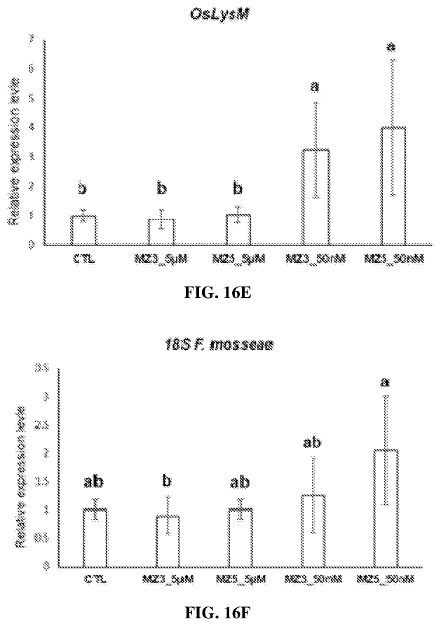

To test if MZ compounds have an impact on AM fungi (AMF), the effect of MZ3 and MZ5 at two concentrations (5 μM and 50 nM) on spores germination of the AMF Gigaspora margarita was examined, using GR24 (10 nM) and acetone as a control. The highest spore germination rate was obtained after GR24 exposure, while 3 and 7 days treatment with MZ3 and MZ5 respectively at both concentrations had no effect (FIGS. 14A-14C). Similarly, Zaxinone did not affect G. margarita spores germination after 3 and 7 days (FIGS. 15A-15B). There was also no alteration in intraradical fungal structures or colonization rate (FIGS. 16A-16C). In line with this result, the expression levels of the AM marker genes OsPt11 and OsLysM, and the fungal housekeeping gene (Fm18S rRNA) did not show any significant difference between control and 5 mM treated plants. The 50 nM treatment even induced a slight upregulation of these AM marker genes (FIGS. 16D-16F). These results show that the two mimics would not have a negative side effect on AM fungi and mycorrhization if applied 10 days after inoculation.

### Example 7. The Compounds does not Affect the Functionality of the Symbiosis

Materials and Methods

Gene Expression Analysis of Mycorrhizal Plants

Total genomic DNA was extracted from F. mosseae sporocarps and O. sativa shoots using the DNeasy Plant Mini Kit (Qiagen), according to the manufacturer's instructions. Genomic DNAs were used to test each primers pair designed for real-time PCR to exclude cross hybridization. Total RNA was extracted from rice roots using the Plant RNeasy Kit (Qiagen), according to the manufacturer's instructions. Samples were treated with TURBO™ DNase (Ambion) according to the manufacturer's instructions. The RNA samples were routinely checked for DNA contamination by means of PCR analysis, using primers for OsRubQ1²⁹ (Guimil et al. 2005). For single-strand cDNA synthesis about 1000 ng of total RNA was denatured at 65° C. for 5 min and then reverse-transcribed at 25° C. for 10 min, 42° C. for 50 min, and 70° C. for 15 min. The reaction was carried out in a final volume of 20 μl containing 10 μM random primers, 0.5 mM dNTPs, 4 μl 5× buffer, 2 μl 0.1 M DTT and 1 μl Super-Script II (Invitrogen). Quantitative RT-PCR (qRT-PCR) was performed using a Rotor-Gene Q 5plex HRM Platform (Qiagen). Each PCR reaction was carried out in a total volume of 15 μl containing 2 μl diluted cDNA (about 10 ng), 7.5 μl 2×SYBR Green Reaction Mix, and 2.75 μl of each primer (3 μM). The following PCR program was used: 95° C. for 90 sec, 40 cycles of 95° C. for 15 sec, 60° C. for 30 sec. A melting curve (80 steps with a heating rate of 0.5° C. per 10 sec and a continuous fluorescence measurement) was recorded at the end of each run to exclude the generation of non-specific PCR products. All reactions were performed on at least three biological and three technical replicates. Baseline range and take off values were automatically calculated using Rotor-Gene Q 5plex software. Transcript level of OsPT11²⁹ (Guimil et al. 2005); OsLysM³⁰ (Fiorilli et al., 2015) and Fm18S³¹ (Balestrini et al., 2007) were normalized using OsRubQ1 housekeeping gene²⁹ (Guimil et al. 2005). Only take off values leading to a Ct mean with a standard deviation below of 0.5 were considered. Statistical tests were carried out through one-way analysis of variance (one-way ANOVA) and Tukey's post hoc test, using a probability level of p<0.05. All statistical elaborations were performed using PAST statistical³² (version 2.16; Hammer et al. 2001).

Statistical tests were carried out through one-way analysis of variance (one-way ANOVA) and Tukey's post hoc test, using a probability level of p<0.05. All statistical elaborations were performed using PAST statistical package (version 2.16; Hammer et al. 2001).

Results

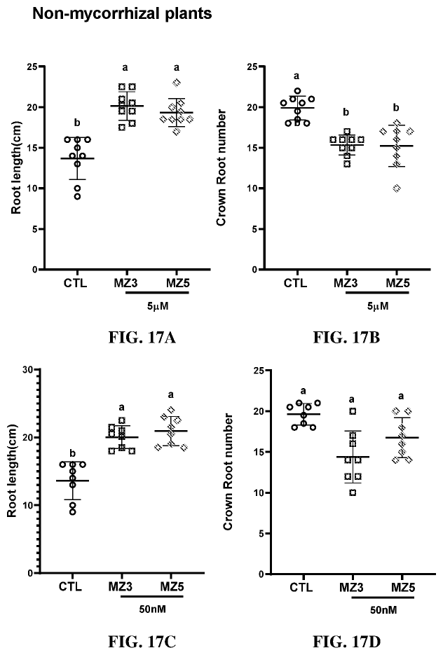

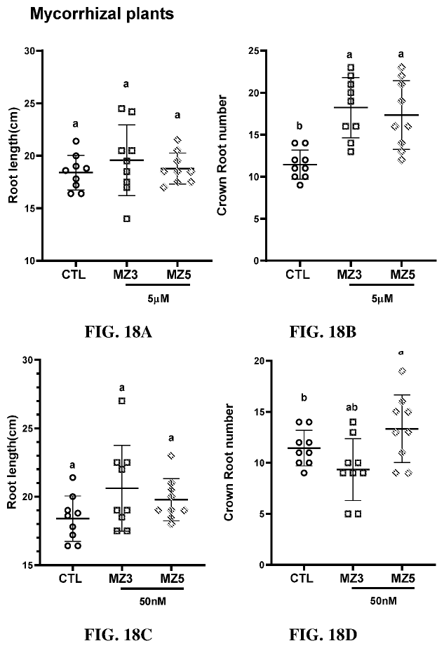

The impact of MZ on the whole colonization process was further investigated to get insights into their effects during symbiotic events. The evaluation of the intensity of mycorrhizal colonization on large lateral roots, the preferential niche for AMF, displayed a decrease of fungal and arbuscule abundance in treated plants at both concentrations (5 μM and 50 nM) compared to controls (FIGS. 16A-16F) while the fungal morphology was maintained (data not shown). The molecular analysis, which considers the whole root apparatus and is based on the evaluation of the transcripts abundance of the plant AM marker genes OsPt11 and OsLysM and the fungal housekeeping gene (Fm18S), showed no differences between control and 5 μM treated plants. In addition, at 50 nM, a slight up-regulation of the plant and fungal marker genes was observed (FIGS. 16A-16F). The non-mycorrhizal and mycorrhizal plants were grown in sand and watered with Long Ashton solution (3.2 μM Pi) for five weeks post AM fungus inoculation. Twice a week treatment with MZ compounds at 5 μM or 50 μM increased the crown root length in non-mycorrhizal plants (FIGS. 17A-17D), and the crown root number in mycorrhizal plants (FIGS. 18A-18D). Considering that MZ led to an increase of the crown root number in mycorrhizal plants (FIGS. 18A-18D), these results show that the fungus is not only localized in large lateral roots but also extends to crown roots. In the whole, these data show that MZ compounds did not influence the establishment and functionality of the AM symbiosis and support their potential eco-friendly application in the field.

MZ compounds act as the promoter of plant growth and development by regulating the level of SLs, which are key regulators in establishing plant architecture, mediator of biotic and abiotic stress responses, and major component for Striga perception. The results demonstrate that MZ compounds have a large agricultural application potential as growth stimulants and a novel tool to combat root parasitic plants, a major threat to global food security, without affecting AM symbiosis. In addition, MZ compounds are helpful for understanding the biology of Zaxinone and the regulation of SL biosynthesis.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

### REFERENCES

1. Chaiwanon, J. et al. (2016). Information Integration and Communication in Plant Growth Regulation. Cell 164, 1257-1268
2. Moise, A. R., Al-Babili, S., and Wurtzel, E. T. (2014). Mechanistic aspects of carotenoid biosynthesis. Chem. Rev. 114, 164-193
3. Cutler, S. R., Rodriguez, P. L., Finkelstein, R. R., and Abrams, S. R. (2010). Abscisic acid: emergence of a core signaling network. Annu. Rev. Plant Biol. 61, 651-679
4. Al-Babili, S., and Bouwmeester, H. J. (2015). Strigolactones, a novel carotenoid-derived plant hormone. Annu. Rev. Plant Biol. 66, 161-186
5. Alder, A. Jamil, M., Marzorati, M., Bruno, M., Vermathen, M., Bigler, P., Ghisla, S., Bouwmeester, H., Beyer, P., and Al-Babili, S. (2012). The path from beta-carotene to carlactone, a strigolactone-like plant hormone. Science 335, 1348-1351
6. Bruno, M. et al. (2017). Insights into the formation of carlactone from in-depth analysis of the CCD8-catalyzed reactions. FEBS lett. 591, 792-800
7. Abuauf, H. et al. (2018). The Arabidopsis DWARF27 gene encodes an all-trans-/9-cis-β-carotene isomerase and is induced by auxin, abscisic acid and phosphate deficiency. Plant Sci. 277, 33-42
8. Booker, J. et al. (2004). MAX3/CCD7 is a carotenoid cleavage dioxygenase required for the synthesis of a novel plant signaling molecule. Curr. Boil. 14, 1232-1238
9. Zhang, Y. et al. (2014). Rice cytochrome P450 MAXI homologs catalyze distinct steps in strigolactone biosynthesis. Nat. Chem. Biol. 10, 1028-1033.
10. Abe, A. et al. (2014). Carlactone is converted to carlactonoic acid by MAXI in Arabidopsis and its methyl ester can directly interact with AtD14 in vitro. Proc. Natl. Acad. Sci. U.S.A. 111, 18084-18089
11. Gomez-Roldan, V. et al. (2008). Strigolactone inhibition of shoot branching. Nature 455, 189-194
12. Umehara, M. et al. (2008). Inhibition of shoot branching by new terpenoid plant hormones. Nature 455, 195-200
13. Bonfante, P., and Genre, A. (2008). Plants and arbuscular mycorrhizal fungi: an evolutionary-developmental perspective. Trends Plant Sci. 13, 492-498
14. Gutjahr, C., and Parniske M. (2013). Cell and developmental biology of arbuscular mycorrhiza symbiosis. Annu. Rev. Cell Dev. Biol. 29, 593-617
15. Fiorilli, V., Wang, J. W., Bonfante, P., Lanfranco, L., and Al-Babili, S. (2019). Apocarotenoids: old and new mediators of the arbuscular mycorrhizal symbiosis. Front. Plant Sci. 10:1186
16. Xie, X. et al. (2010). The strigolactone story. Annu. Rev. Phytopathol. 48, 93-117
17. Parker, C. (2012). Parasitic Weeds: A World Challenge. Weed Sci. 60, 269-276
18. Pennisi, E. (2010). Armed and dangerous. Science 327, 804-805
19. Wang, J. Y., Haider, I., Jamil, M., Fiorilli, V., Saito, Y. et al. (2019). The Apocarotenoid Metabolite Zaxinone Regulates Growth and Strigolactone Biosynthesis in Rice. Nat. Commun. 10, 810.
20. Jia, K., Dickinson, A. J., Mi, J., Cui, G., Kharbatia, N. M., Guo, X., et al. (2019). Anchorene is an endogenous diapocarotenoid required for anchor root formation in Arabidopsis. Sci. Adv. in press. doi: 10.1101/496737
21. Dickinson, A. J. et al. (2019). β-cyclocitral is a conserved root growth regulator. Proc. Natl. Acad. Sci. U.S.A.
22. Jamil, M., Kountche, B. A., Haider, I., Guo, X., Ntui, V. O., Jia, K.-P., et al. (2018). Methyl phenlactonoates are efficient strigolactone analogs with simple structure. J. Exp. Bot. 69, 62319-62331. doi: 10.1093/jxb/erx438.
23. Al-Babili, S., Jamil, M., Kountche, B. A., Haider, I., Wang, J. Y., Aldossary, F., Zarban, R. A., et al (2019). Methylation at the C-Yin D-ring of Strigolactone Analogs Reduces Biological Activity in Root Parasitic Plants and Rice. Front. Plant Sci. 10, 353.
24. Jami, M. et al. (2012). Striga hermonthica parasitism in maize in response to N and P fertilisers. Field Crops Res. 134, 1-10
25. Boubacar, A. K. et al. (2019). Suicidal germination as a control strategy for Striga hermonthica (Benth.) in smallholder farms of sub-Saharan Africa. Plants, People, Planet doi.org/10.1002/ppp3.32
26. Livak, K. J. and Schmittgen, T. D. (2001). Analysis of relative gene expression data using real-time quantitative PCR and the 2^{ΔΔ}CT Method. Methods 25 (4) 402-408.
27. Hewitt, E. J. (1966). Sand and water culture methods used in the study of plant nutrition. Farnham Royal, UK: Commonwealth Agricultural Bureaux.
28. Trouvelot, A., et al. in Mycorrhizae. Physiology and Genetics (eds Gianinazzi-Pearson, V. & Gianinazzi, S.) 217-221 (INRA Press, Paris, 1986)
29. Guimil, S., Chang, H. S., Zhu, T., Sesma, A., Osbourn, A., Roux, C., Ioannidis, V., Oakeley, E. J., Docquier, M., Descombes, P. et al. (2005). Comparative transcriptomics of rice reveals an ancient pattern of response to microbial colonization. Proc. Natl Acad. Sci. USA 102: 8066-8070.
30. Fiorilli, V. et al. (2015). Host and non-host roots in rice: cellular and molecular approaches reveal differential responses to arbuscular mycorrhizal fungi. Front Plant Sci 6, 636.
31. Balestrini, R., Go'mez-Ariza, J., Lanfranco, L., Bonfante, P. (2007). Laser microdissection reveals that transcripts for five plant and one fungal phosphate transporter genes are contemporaneously present in arbusculated cells. Mol. Plant Microbe. In. 20: 1055-1062.
32. Hammer, Ø., Harper, D. A. T., and Ryan, P. D. (2001). PAST: paleontological statistics software package for education and data analysis. Palaeontol. Electron. 4, 9.

1. A compound having a structure of Formula I: 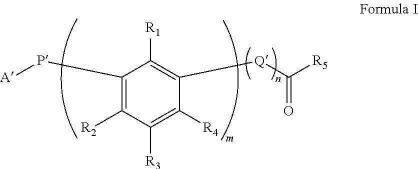
(a) wherein A′ is an unsubstituted alkyl group, a substituted alkyl group, an unsubstituted cycloalkyl group, a substituted cycloalkyl group, an unsubstituted heteroalkyl group, a substituted heteroalkyl group, an unsubstituted cycloheteroalkyl group, a substituted cycloheteroalkyl group, an unsubstituted alkenyl group, a substituted alkenyl group, an unsubstituted heteroalkenyl group, a substituted heteroalkenyl group, a substituted alkynyl group, a substituted heteroalkynyl group, an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group;
(b) wherein P′ is an oxygen atom, a sulfur atom, or absent;
(c) wherein m is zero or a positive integer;
(d) wherein Q′ is —CR₆═CR₇—;
(e) wherein n is zero or a positive integer; and
(f) wherein R₁-R₇, when present, are independently
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,
an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonyl group containing one substituent, a carbonate ester group containing one substituent, an ether group containing one substituent, an aminooxy group optionally containing one or two substituents at the amino nitrogen, or a hydroxyamino group optionally containing one or two substituents,
wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

2. The compound of claim 1, wherein A′ is an unsubstituted aryl group, a substituted aryl group, an unsubstituted heteroaryl group, a substituted heteroaryl group, an unsubstituted polyaryl group, a substituted polyaryl group, an unsubstituted polyheteroaryl group, or a substituted polyheteroaryl group.

3. The compound of claim 1 having a structure of Formula II: 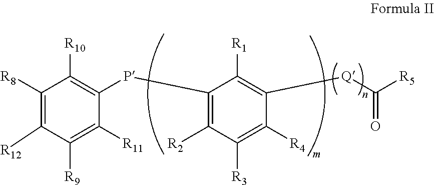
(a) wherein P′, Q′, R₁-R₇, m, and n are as defined in the base claims; and
(b) wherein R₈-R₁₂ are independently
a hydrogen atom, a halogen atom, a sulfonic acid, an azide group, a cyanate group, an isocyanate group, a nitrate group, a nitrile group, an isonitrile group, a nitrosooxy group, a nitroso group, a nitro group, an aldehyde group, an alkoxy group, an acyl halide group, a carboxylic acid group, a carboxylate group, an unsubstituted alkyl group, a substituted alkyl group, an unsubstituted heteroalkyl group, a substituted heteroalkyl group, an unsubstituted alkenyl group, a substituted alkenyl group, an unsubstituted heteroalkenyl group, a substituted heteroalkenyl group, an unsubstituted alkynyl group, a substituted alkynyl group, an unsubstituted heteroalkynyl group, a substituted heteroalkynyl group, an unsubstituted aryl group, a substituted aryl group, an unsubstituted heteroaryl group, a substituted heteroaryl group,
an amino group optionally containing one or two substituents at the amino nitrogen, an ester group containing one substituent, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, a carbonyl group containing one substituent, a carbonate ester group containing one substituent, an ether group containing one substituent, an aminooxy group optionally containing one or two substituents at the amino nitrogen, or a hydroxyamino group optionally containing one or two substituents,
wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

4. The compound of claim 3, wherein R₁₂ is a hydrogen atom, an aldehyde group, an alkoxy group,
an amino group optionally containing one or two substituents at the amino nitrogen, a hydroxyl group optionally containing one substituent at the hydroxyl oxygen, a thiol group optionally containing one substituent at the thiol sulfur, a sulfonyl group containing one substituent, an amide group optionally containing one or two substituents at the amide nitrogen, an azo group containing one substituent, an acyl group containing one substituent, an ether group containing one substituent, an aminooxy group optionally containing one or two substituents at the amino nitrogen, or a hydroxyamino group optionally containing one or two substituents,
wherein the substituents are optionally substituted alkyl groups, optionally substituted heteroalkyl groups, optionally substituted alkenyl groups, optionally substituted heteroalkenyl groups, optionally substituted alkynyl groups, optionally substituted heteroalkynyl groups, optionally substituted aryl groups, optionally substituted heteroaryl groups, or combinations thereof.

5. The compound of claim 3, wherein: (a) R₁₂ is a hydrogen atom, a hydroxyl group, an alkoxy group, an ether group, or a thiol group: (b) R₈-R₁₁ are independently a hydrogen atom, an unsubstituted alkyl group, a substituted alkyl group, an unsubstituted heteroalkyl group, or a substituted heteroalkyl group; or (c) R₈ and R₉ are hydrogen and R₁₀-R₁₁ are independently an unsubstituted alkyl group, a substituted alkyl group, an unsubstituted heteroalkyl group, or a substituted heteroalkyl group.

6. The compound of claim 1, wherein: (a) P′ is an oxygen atom; (a) P is absent.

7. The compound of claim 6, wherein the m and n are individually positive integers; m is 1 and n is 1; m is a positive integer and n is zero or 1; or m is zero and n is a positive integer

8. (canceled)

9. (canceled)

10. (canceled)

11. (canceled)

12. The compound of claim 1, wherein: (a) R₁-R₇, when present, are independently a hydrogen atom, an unsubstituted alkyl group, a substituted alkyl group, an unsubstituted heteroalkyl group, or a substituted heteroalkyl group; or (b) R₁-R₄ are hydrogen and R₅ is an unsubstituted alkyl group, a substituted alkyl group, an unsubstituted heteroalkyl group, or a substituted heteroalkyl group.

13. (canceled)

14. (canceled)

15. (canceled)

16. A compound having a structure of:

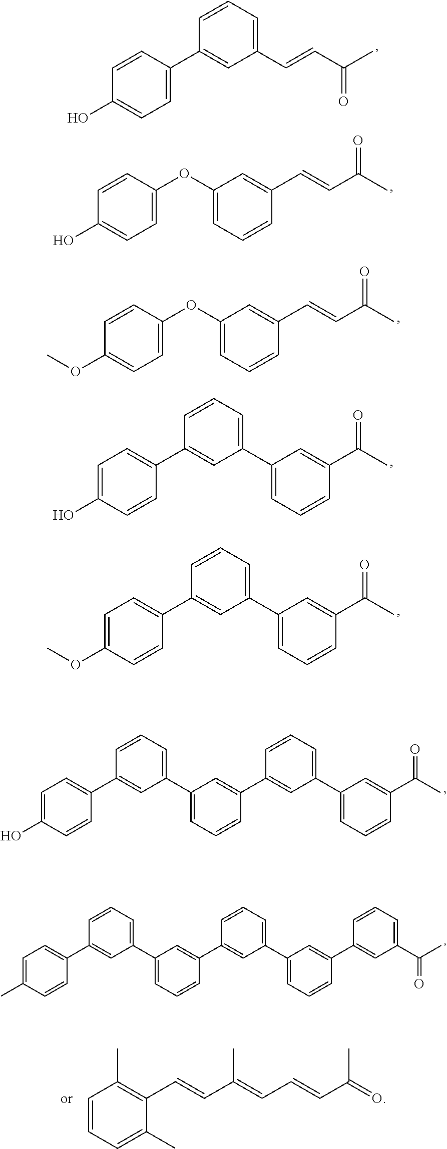

17. A formulation comprising
(a) one or more compounds of claim 1, or salts thereof, and
(b) one or more formulation excipients, optionally, wherein the formulation is in the form of dusting powders, gels, wettable powders, water-dispersible granules, water-dispersible tablets, effervescent compressed tablets, emulsifiable concentrates, microemulsifiable concentrates, oil-in-water emulsions, oil flowables, aqueous dispersions, oil dispersions, suspoemulsions capsule suspensions, emulsifiable granules, soluble liquids, water-soluble concentrates (with water or a water miscible organic solvent as carrier), or impregnated polymer films.

18. The formulation of claim 17, wherein the excipient is a solid carrier, a liquid carrier, or a surface-active agent.

19. (canceled)

20. A method for promoting plant growth comprising:
(i) applying one or more formulations to a plant, a plant part, or a growing site of plant, wherein the one or more formulations comprise
(a) one or more compounds of claim 1, or salts thereof, and
(b) one or more formulation excipients,
wherein the one or more compounds or salts thereof are in an effective amount to promote plant growth.

21. The method of claim 20, wherein the plant is a cereal, grain, or vegetable plant.

22. (canceled)

23. The method of claim 20, wherein: (a) the plant part is seed or seedling: (b) the growing site of plant is soil before, during, or after planting the plant: or (c) the growing site of plant is a growth medium; and optionally, the method further comprising a step of diluting the formulation in a liquid carrier prior to step (i).

24. (canceled)

25. (canceled)

26. (canceled)

27. The method of claim 23, wherein the liquid carrier is water, and optionally, wherein the concentration of the one or more compounds or salts thereof after dilution is between about 0.1 μM and about 1 mM.

28. (canceled)

29. The method of claim 20, wherein the one or more compounds or salts thereof are in an effective amount to decrease biosynthesis and release of strigolactone plant hormones from the plant, optionally, wherein the biosynthesis and release of strigolactone plant hormones from the plant is decreased by at least about 40% compared to an untreated plant under same conditions and optionally, the method further comprising applying one or more agriculturally beneficial agents prior to, during, or after step (i).

30. The method of claim 29, wherein the one or more agriculturally beneficial agents are fertilizers, micronutrients, microorganisms, or a combination thereof.

31. (canceled)

32. (canceled)

33. A method for inhibiting growth of a parasitic weed comprising:
(i) applying one or more formulations to the parasitic weed, a part of the parasitic weed, or a growing site of parasitic weed
wherein the one or more formulations comprise
(a) one or more compounds of claim 1, or salts thereof, and
(b) one or more excipients,
wherein the one or more compounds or salts thereof are in an effective amount to inhibit growth of the parasitic weed.

34. The method of claim 33, wherein: (a) the parasitic weed is a Striga species or an Orobanche species of the Orobanchaceae family: (b) the growing site of the parasitic weed is soil before, during, or after planting a host plant; or (c) the one or more compounds or salt thereof are in an effective amount to inhibit germination of a seed of the parasitic weed and optionally, the method further comprising applying one or more herbicides prior to, during, or after step (i), or a step of diluting the formulation in a liquid carrier prior to step (i).

35. (canceled)

36. The method of claim 34, wherein the growing site of the parasitic weed is soil after planting the host plant; optionally, wherein the liquid carrier is water, and optionally, wherein the concentration of the one or more compounds or salt thereof after dilution is between about 0.1 μM and about 1 mM, preferably between about 1 μM and about 10 μM.

37. (canceled)

38. (canceled)

39. (canceled)

40. (canceled)

41. (canceled)
